(19) United States
(12) Patent Application Publication
(10) Pub. No.: US 2014/0161826 A1
(43) Pub. Date: Jun. 12, 2014

(54) NON-SELF T-CELL EPITOPE FUSED TO AN ANTIBODY THAT RECOGNISES A TUMOUR-SPECIFIC CELL-SURFACE RECEPTOR AND USES THEREOF

(71) Applicant: Antonello Pessi, Pomezia (IT)

(72) Inventor: Antonello Pessi, Pomezia (IT)

(21) Appl. No.: 14/110,122

(22) PCT Filed: Apr. 5, 2012
(86) PCT No.: PCT/EP2012/056378

§ 371 Date: Dec. 23, 2013

Related U.S. Application Data
(60) Provisional application No. 61/472,851, filed on Apr. 7, 2011.

(30) Foreign Application Priority Data
Apr. 14, 2011 (GB) 1106357.5

(51) Int. Cl. A61K 47/42; A61K 39/245; A61K 39/12; A61K 39/165; A61K 39/205; A61K 39/145; A61K 39/29

(52) U.S. Cl. CPC A61K 47/42; A61K 39/145; A61K 39/245; A61K 39/29; A61K 39/292; A61K 39/165; A61K 39/205; A61K 39/12
USPC 424/174.1; 424/184.1; 424/209.1; 424/230.1; 424/228.1; 424/227.1; 424/212.1; 424/224.1; 424/218.1; 424/130.1; 424/277.1

(57) ABSTRACT

A first composition comprising a ligand part being able to specifically bind to a target, the target being on a diseased cell of an organism, and an epitope part having a non-self epitope, or encoding a non-self epitope, the non-self epitope not naturally being encoded by the organism, wherein the target is internalized after forming a complex with the composition and the non-self epitope is presented on the cell surface of said diseased cell of an organism after internalization.

The present invention relates to compositions suitable for use in prevention and treatment of disease and uses thereof.

### INTRODUCTION

Cancer is still a largely unmet medical need and the leading cause of death in industrialized countries. The immune system, if appropriately activated, has immense potential as it is evident from mismatched transplanted organs undergoing rapid immunological attack and rejection. The immune system can recognize and kill pre-cancer and cancer cells. However, the development of immune strategies for cancer therapy has been associated until now with limited success.

The development of cancer vaccines is still characterized by many hurdles and challenges. Cancer vaccines are universally based on the notion of tumour associated antigens (TAA).

Tumor antigens are molecules that
1) are often present at higher concentration in cancer cells with respect to normal tissues, and are in general self antigens;
2) play a crucial role in the generation, survival or progression of the cancer cells.

The mechanism of action of present candidate cancer vaccines are believed to be based on:
1) induction of immunological response against TAA;
2) interaction between tumor antigen specific antibodies or tumor antigen specific T cells and cancer cells triggering a chain of molecular reactions ending in the death of the cancer cell.

The major limitation of this approach is linked to the inherent restriction of T-cell recognition to non-self antigens. While effector T-cells are efficient in the rejection of large organs and foreign tissues, the wide application of tumor-specific T-cells for cancer therapy is limited because tumor antigens are “encoded” by the organism carrying the cancer: they are self antigens. Typically the immunological system does not respond readily to self antigens because of the so called “tolerance”, and vaccination with self-antigens generally leads to the activation of a T cell repertoire with low avidity for the antigen or with non-functional phenotype. This presents a challenge for cancer vaccines [1].

It is known that tumor cells can become a better inducer and finally target of T cell immune attack if the rate and the extent of antigen presentation on their surface is artificially increased. [2].

A recent report suggests that the induction of an adaptive immune response is an important mechanism contributing to the overall efficacy of therapeutic antibodies like Herceptin (Trastuzumab) in cancer [3]. The mechanism responsible for induction of an anti-tumor adaptive immune response during treatment with the tumor specific antibodies is believed to be based on the mechanism of intracellular processing. During treatment of HER-2 positive tumors with the Herceptin antibody, tumor cells become the target of HER-2 specific T cells [4-5]. In presence of the sensitizing antibody, HER2-reactive low avidity T-cell clones, generated from healthy donors and breast cancer patients, have been shown to kill class I-matched, HER-2 over-expressing tumor cells [6]. Furthermore, the combination therapy of anti-HER-2 antibody and vaccination with HER-2 derived peptides has been tested in a phase-I clinical trial [7]. Enhancement of specific CD8 T cell recognition of EphA2 positive tumours occurs both in vitro and in vivo after treatment with EphA2 ligand agonist. Complete eradication of tumours in SCID mice has been obtained by combining passive transfer of EphA2 specific T cells of modest functional activity with the administration of agonist ligand or agonist anti-EphA2 antibody [8]. Antibodies fused with additional immunogenic sequences have been disclosed at the 24th and 25th Annual Meeting of the International Society for Biological Therapy (CDX-1307 and CDX-1401) [9-11].

The present invention relates to methods for the treatment of diseases, including but not limited to cancer.

### STATEMENTS OF INVENTION

The present invention relates to:

A first composition comprising:

a ligand part being able to specifically bind to a target, the target being on a diseased cell of an organism, and
an epitope part having a non-self epitope, or encoding a non-self epitope, the non-self epitope not naturally being encoded by the organism, wherein
the target is internalized after forming a complex with the composition and the non-self epitope is presented on the cell surface of said diseased cell of an organism after internalization

A first composition as disclosed herein in combination with an immunogenic second composition comprising an epitope which is identical to a non-self epitope of the first composition, or comprising an epitope which elicits an immune response which is cross-reactive with the non-self epitope of the first composition.

A first composition as disclosed herein for use in the treatment of disease, or for use in the preparation of a medicament for the treatment of disease, and wherein the first composition is for use or used in conjunction together with an immunogenic second composition comprising an epitope which is identical to a non-self epitope of the first composition, or comprising an epitope which elicits an immune response which is cross-reactive with the non-self epitope of the first composition.

A method of treating disease by administering to an organism
a) a first composition as disclosed herein and optionally
b) an immunogenic second composition comprising an epitope which is identical to a non-self epitope of the first composition, or comprising an epitope which elicits an immune response which is cross-reactive with the non-self epitope of the first composition.

A kit of parts comprising:

a) a first composition as disclosed herein; and
b) a immunogenic second composition comprising an epitope which is identical to a non-self epitope of the first composition, or comprising an epitope which elicits an immune response which is cross-reactive with the non-self epitope of the first composition.

A first composition as disclosed herein wherein the epitope part and ligand part are comprised within the same cell or virus.

A first composition as disclosed herein wherein the epitope part comprises a nucleic acid which encodes a non-self epitope.

A first composition for use in the treatment of disease, wherein the conjugate comprises
a ligand part being able to specifically bind to a target, the target being on a diseased cell of an organism, and
an epitope part having a non-self epitope not naturally being encoded by the organism, wherein
   the target is internalized after forming a complex with the composition and the non-self epitope is presented on the cell surface of said diseased cell of an organism after internalization,
   and wherein the composition is used in conjunction with an agent that is specific for the presented non-self epitope, such as an antibody or T cell population.

A method of medical treatment comprising delivering to an individual in need thereof an effective amount of a first composition as disclosed herein in conjunction with an agent that is specific for the presented non-self epitope, such as an antibody or T cell population.

An immunogenic composition comprising a non-self antigen for use in the prevention or treatment of cancer.

An immunogenic composition comprising an antigen which is not a tumour associated antigen for the prevention or treatment of cancer.

An immunogenic composition comprising an antigen which is not associated with any human or non human animal disease, for the prevention or treatment of human or non-human animal disease.

A kit of parts comprising
a) a first composition comprising:
a ligand part being able to specifically bind to a target, the target being on a diseased cell of an organism, and
an epitope part having a non-self epitope not naturally being encoded by the organism, wherein
the target is internalized after forming a complex with the composition and the non-self epitope is presented on the cell surface of said diseased cell of an organism after internalization; and
b) a second immunogenic composition comprising an epitope which is identical to a non-self epitope of the first composition, or comprising an epitope which elicits an immune response which is cross-reactive with the non-self epitope of the first composition;

Use of a first composition in the preparation of a medicament for the prevention or treatment of a disease, or for the use in the prevention or treatment of a disease, wherein the composition comprises:
a ligand part being able to specifically bind to a target, the target being on a diseased cell of an organism, and
   an epitope part having a non-self epitope not naturally being encoded by the organism, wherein the target is internalized after forming a complex with the first composition and the non-self epitope is presented on the cell surface of said diseased cell of an organism after internalization,
   wherein the first composition is delivered to an individual with a pre-existing immune response to the epitope when presented after internalisation.

The first composition may be a ligand-epitope conjugate, the ligand-epitope conjugate comprising: a ligand part being able to specifically bind to a target, the target being on a diseased cell of an organism, and an epitope part having a non-self epitope not naturally being encoded by the organism, wherein the target is internalized after forming a complex with the conjugate and the non-self epitope is presented on the cell surface of said diseased cell of an organism after internalization.

### FIGURES

FIG. 1 discloses a schematic of ligand induced intracellular processing of antigens in cancer. A) Display of tumour associated antigen (TAA) epitopes in cancer cell B) Display of TAA epitopes can be increased by ligand induced internalization of the tumor antigen C) ligand induced presentation of a non-self antigen by tumor cells.

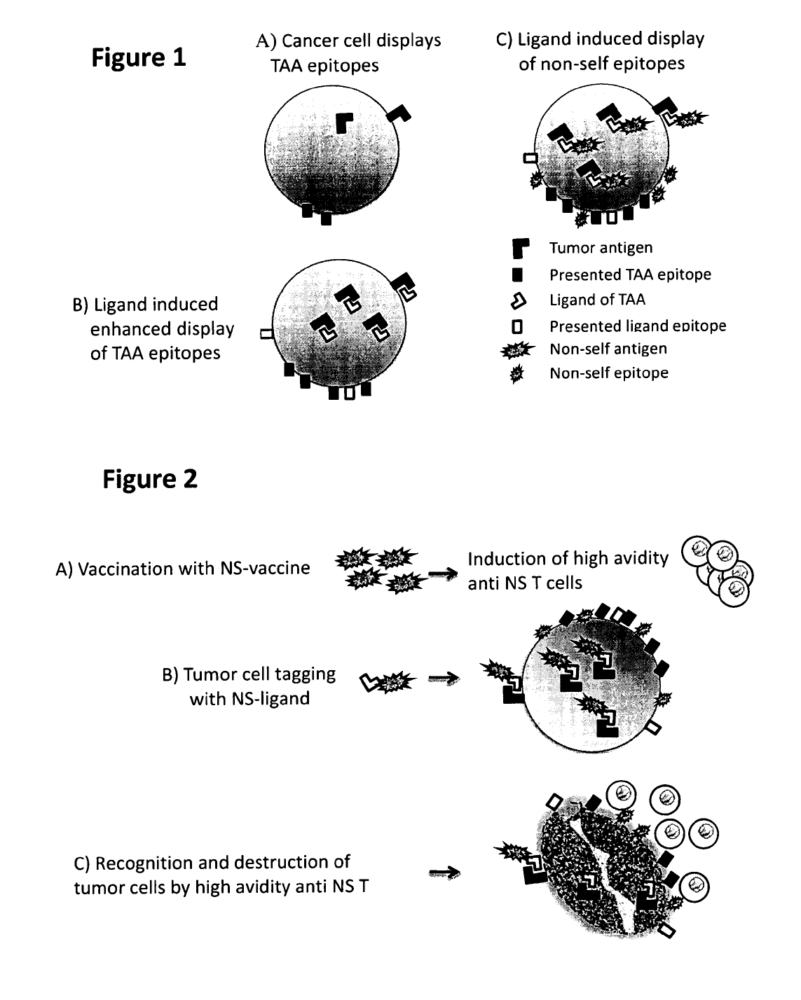

FIG. 2: Schematic of the novel two components treatment: A) NS-vaccination. B) Cancer cells-tagging with NS-ligand. C) Cancer cells killing by anti-NS T cells.

FIG. 3: Analysis of purified mAbs by SDS-PAGE under reducing conditions. Lane 1: standard, Lane 2-4: purified nnAb15, nnAb15-OVAPep2 and nnAb15-OVAPep1.

FIG. 4: EphA2 expression in human and mouse cancer cell lines. Western Blot analysis with EphA2 specific antibody on total cell lysate from MC38, HCT116, TUBO, 4T1 cells. α-actin antibody confirmed equal loading of proteins.

FIG. 5: Binding curve obtained by ELISA assays of purified nnAb15 (), mAb15-OVAPep2 (▪) and nnAb15-OVAPep1 (▴) to murine C35 Luc (A), MC38 (B) and human (EBNA-293/EphA2) EphA2 positive cells (C).

FIG. 6: Analysis by Flow Cytometry of OVA257-264/Kb complex presentation on tumor cells surface. EphA2 positive cells (C35 luc) were treated for 48 hrs at 37° C. with increasing concentration of anti-EphA2 mAbs and stained with specific 25-D1.16 PE conjugated antibody. As positive control cells were pulsed with SIINFEKL peptide (2 μM) for 1 h at 37° C.

FIG. 7: FNγ ELIspot on splenocytes from C57BL/6 mice immunized with Ad5OVA TAG (10̂8 vp). Splenocytes were harvested at 10 (A) or 24 days (B) post immunization and restimulated with C35Luc cells pretreated (48 hrs) with OVA-mAbs at increasing concentration or with SIINFEKL peptide (1 h). Responses are reported as the number of antigen specific IFNγ spot forming cells/million of splenocytes. Each bar represents the mean of quadruplicate values.

FIG. 8: Analysis of purified mAbs by SDS-PAGE under reducing conditions. Lane 1: standard, Lane 2,3: purified Herceptin and Herceptin-OVAPep2.

FIG. 9: Binding curve obtained by ELISA assays of purified Herceptin () and Herceptin-OVAPep2 (A) to murine C35 Luc (A), MC38 (B) and human SKBR3 (C) ErbB2 positive cells.

FIG. 10: Analysis by Flow Cytometry of OVA257-264/Kb complex presentation on tumor cells surface. ErbB2 positive cells C35 Luc (A) and MC38 (B) were treated for 48 hrs at 37° C. with anti-ErbB2 mAbs (4 μM) and stained with specific 25-D1.16 PE conjugated antibody. As positive control cells were pulsed with SIINFEKL peptide (2 μM) for 1 h at 37° C.

FIG. 11: FNγ ELIspot on splenocytes from C57BL/6 mice immunized with Ad5OVA TAG (10̂8 vp). Splenocytes were harvested at 24 days post immunization and restimulated with C35 Luc cells pretreated (48 hrs) with OVA-mAbs at increasing concentration or with SIINFEKL peptide (1 h). Responses are reported as the number of antigen specific IFNγ spot forming cells/million of splenocytes. Each bar represents the mean of quadruplicate values.

FIG. 12: Vector mediated presentation of OVA epitope

### DETAILED DESCRIPTION

The present invention relates generally to a method for prevention or treatment of a disease, the method using a non-self antigen, to induce a tolerance-insensitive immune response, and a ligand having specificity for a specific receptor, the latter allowing targeting of the tolerance insensitive immune response to specific cells.

It is known that Ligands bind to cognate receptors (R) forming a complex (LR complex), which may be internalized and directed towards the intracellular degradation pathway where the complex is fragmented into peptides. These peptides in turn, are presented on the cell surface in a complex with MHC class I or class II. Therefore the administration of a ligand L to a cell expressing the cognate receptor R, leads to enhanced proteosomal processing of the ligand and consequently to an increase in the concentration of LR derived peptides presented on the surface of cell, such as a cancer cell.

In the present invention cells are targeted for treatment by delivery of a first composition such as a ligand-epitope conjugate to a cell, which composition e.g. conjugate is taken up, and then all or part of which is processed and presented by the cell. Presentation of the epitope targets the cell for treatment, which may be effected by the immune response, or other specific means capable of binding the presented epitope. In particular, where the ligand binds to a self-target and the epitope is a non-self epitope, an immune response generated against the presented non-self epitope is not affected by tolerance.

An immune response may be generated to the presented, non-self, epitope by the delivery of an immunogenic composition comprising the non-self antigen, or mimetope thereof. Where the immunogenic second composition is delivered to an individual before the first composition, the individual may comprise a population of immune cells, such as T cells, which are ready to react with the presented epitope. In essence the individual is vaccinated against the non-self epitope.

The invention also relates to a use or method as disclosed herein wherein the immunogenic second composition is delivered after the first composition, or the first composition is delivered concomitantly, or substantially concomitantly, such as on the same day as the immunogenic second composition.

In the present invention the specificity of the immune response to target cells is controlled by the ligand binding and potentially may also be modulated at the level of interaction of T cells with the processed and presented epitope.

The present invention discloses a composition comprising:

a ligand part being able to specifically bind to a target, the target being on a diseased cell of an organism, and
an epitope part having a non-self epitope, or encoding a non-self epitope, the non-self epitope not naturally being encoded by the organism, wherein
the target is internalized after forming a complex with the composition and the non-self epitope is presented on the cell surface of said diseased cell of an organism after internalization.

The composition may also be referred to as a first composition herein.

The ligand part and epitope part of the composition are suitably associated with each other such that binding of the ligand to the diseased cell allows for the epitope part to be internalised into the diseased cell and then the epitope to be presented on the cell surface.

In one aspect the first composition may comprise a ligand epitope conjugate, the conjugate comprising a ligand part and an epitope.

The ligand part of said conjugate may be conjugated to the epitope part by chemical cross linking. Alternatively the conjugate may be a fusion protein comprising the ligand part and the epitope part. In another embodiment the parts may be associated by protein-protein interactions, such as leucine zippers. The ligand part and epitope part may be joined directly or through a linker.

In another embodiment the conjugate may also be a single molecule in which the epitope forms a part of the whole ligand, for example where the ligand is synthetic and/or where the ligand is a non-self antigen. Thus the conjugate of the invention suitably minimally comprises dual functionalities, namely the ability to bind to a target and an epitope that can be presented by a target cell, but does not necessarily require any specific conjugation activity or process be undertaken to link or otherwise associate the 2 different elements of the conjugate. The conjugation of the ligand to the non-self antigenic sequence suitably does not interfere with receptor binding, internalization and proteosomal degradation.

In one aspect the composition may comprise a ligand and epitope that are associated by means other than conjugation. For example, the epitope part and ligand part may be comprised within the same organism, such as cell or virus. In this aspect the virus or cell provides both the ligand and epitope parts. For example, the ligand and epitope part may form 2 elements of a virus, either naturally occurring or genetically engineered, which virus can bind to a target on a diseased cell. The virus can comprise non-self epitopes or encode them, such that, as a result of binding of the virus to the cell, the non-self epitope has the capacity to be presented on the surface of the diseased cell. In one aspect the virus may be an adenovirus.

Therefore in one aspect the ligand part may be a peptide, a protein, an antibody or a viral vector, capable of interacting with a target (e.g. a receptor) present on the surface of the target cell, and of being internalized.

Therefore, in one aspect the epitope part may comprise a nucleic acid species, such as DNA or RNA, which encodes a non-self epitope. In this case the presentation of the non-self epitope suitably occurs after transcription and/or translation of the nucleic acid species within the diseased cell. The nucleic acid may be directly linked or conjugated to the ligand or associated such as by being a part of the same species or organism.

In one aspect the epitope part is a peptide or a string of peptides, or a protein.

In one aspect the first composition does not comprise a translocation domain.

The target for the ligand may be a receptor or antigen on a cell. In one embodiment the target is specific to a diseased cell, such as a tumour cell, or has an expression pattern which is different from a wild type cell, such as having a particular antigen over-expressed.

In one aspect the receptor is tumor-specific. One class of cell surface tumor antigens that may be suitable targets are the family of receptor tyrosine kinases (RTK) [2]. RTK are commonly expressed by a broad range of cancer types, where they contribute to disease progression, providing proliferative, survival, angiogenic and migration benefit to cancer cells. Given RTK cancer potentiating functions, they are popular targets for anticancer therapies designed to block signalling cascade and to reduce their expression levels. Under normal conditions of ligand/RTK binding, the receptor dimerizes in the cell membrane and becomes autophosphorylated on cytoplasmic tyrosine residues. Phosphorylated RTK are then competent to interact with adaptor proteins, such as E3-ligase, resulting in ubiquitinilation and internalization via clathrin-coated pits. Endocytosed RTK may become substrates for proteosomal degradation. Among RTK, experimental data support the choice of Her2/neu and EphA2 as suitable receptor because ligand induced intracellular processing has been demonstrated. Other RTK, like EGFR, c-Met, VEGFR1 and VEGFR2 are also suitable target because ligand induced receptor degradation has been demonstrated for these receptors in cancer cells.

The cellular target for the ligand may therefore comprise a receptor tyrosine kinase selected from the group consisting of:

The ErbB or Epidermal Growth Factor (EGF) family of receptor tyrosine kinases (RTKs) such as: EGF R/ErbB1/HER1, ErbB2/Neu/HER2, ErbB3/HER3, and ErbB4/HER4;
The EPH family of receptor tyrosine kinases (EPHA1, EPHA2, EPHA3, EPHA4, EPHA5, EPHA6, EPHA7, EPHA8, EPHA9, EPHA10; EPHB1, EPHB2, EPHB3, EPHB4, EPHB5, EPHB6)
The c/MET family (Met and RON) and the VEGFR-1 (Flt-1) VEGFR-2 (KDR/Flk-1). VEGFR-3(Flt-4)

Moreover, other classes of receptor over-expressed in diseases such as cancer and showing a similar pathway of internalization-induced degradation may also be used in the present invention.

The target may be a presented antigen in the context of an MHC molecule.

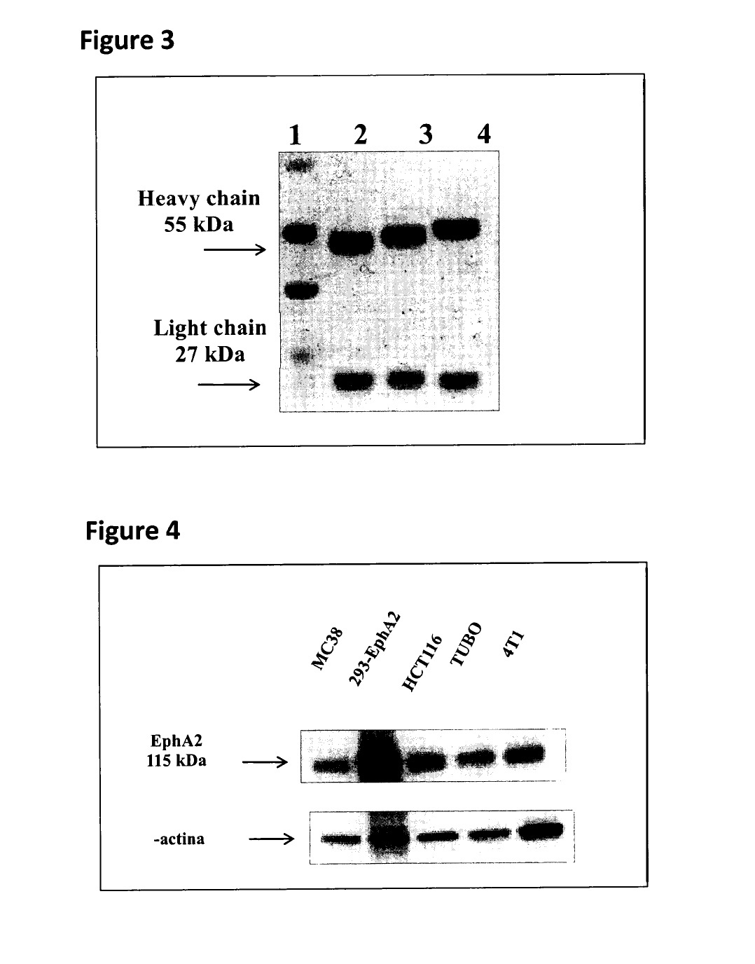

To identify cell surface molecules capable of binding the non self (NS) ligand and of delivering the NS-ligand into the target cell, internalization and degradation assays can be performed. In one embodiment the assay is as follows: cells expressing the receptor are incubated with control ligand or the NS-ligand at different concentrations (e.g. from 1 to 100 μg/mL) for different times (e.g. 1, 2, 4 and 6 hours). Cells are then lysed in TPER buffer (Pierce), 10 minutes at 4 degrees C. 30 μg of cell lysate is loaded onto an SDS PAGE gel and after electrophoresis, proteins are transferred onto nitrocellulose, and probed with primary antibodies against the receptor using standard techniques. Secondary antibodies conjugated to HRP or AP can be used as developing reagents. The amount of receptor found on the cell upon NS-ligand treatment are quantified by densitometric scanning. As an example of this type of assay see FIG. 2 in: Ansuini H et al. Anti-EphA2 Antibodies with Distinct In Vitro Properties Have Equal In Vivo Efficacy in Pancreatic Cancer. J Oncol. 2009; 2009:951917.

The ligand part of said first composition, eg conjugate is any component that is capable of directing specific binding of the first composition, eg conjugate to a target of interest. It may be an antibody or an antigen-binding fragment of an antibody, especially where the target is an antigen on the surface of a cell.

Antibody binding fragments can include polyclonal and monoclonal antibodies and antigen-binding derivatives or fragments thereof. Antibodies may be of any class such as IgG, IgM, IgA, IgE or IgD. Well known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art.

In more detail, reference to antibodies includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies generally lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Thus, antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen. Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. While both polyclonal and monoclonal antibodies can be used in the methods described herein, it is preferred that a monoclonal antibody is used where conditions require increased specificity for a particular protein.

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanised antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:77 O-7 '83 (1992)).

Where the target is a receptor the ligand part may also be the natural ligand for the receptor or a receptor binding fragment thereof; it may be a mimic of the natural ligand, such as peptide or synthetic molecule that is able to bind the receptor; or it may be a non-natural ligand or mimic thereof capable of binding to the receptor. An example of the latter includes bacterial protein Internalin B from Listeria binding to the host receptor tyrosine kinase Met.

The ligand part of the first composition (eg ligand epitope conjugate) may be one of: EGF, TGF-a, HB-EGF, amphiregulin, betacellulin, epigen, epiregulin, neuregulin 1, 2, 3, 4, EFNA1, 2, 3, 4, 5, EFNB1, 2, 3, Hepatocyte growth factor (HGF), macrophage stimulating protein (MSP), VEGF-A, VEGF-C and VEGF-D. Other ligands for cellular receptors are well known, in particular those receptors associated with cancerous phenotypes.

The ligand part of the first composition (eg ligand epitope conjugate) in one embodiment is able to induce internalization of a RTK receptor as disclosed herein such as one of the following RTK receptors: Her2/neu, EphA2, EGFR, c-Met, VEGFR1 and VEGFR2, or any other receptor for which over-expression in cancer and ligand induced internalization and degradation is demonstrated.

The ligand part of the first composition (eg ligand-epitope conjugate) is able to specifically bind to a target. The phrases “specifically binds to”, “specific for” or “specifically immunoreactive with”, when referring to an antibody or ligand target binding, generally refers to a binding reaction which is determinative of the presence of the target, such as a polypeptide in the presence of a heterogeneous population of other possible targets such as proteins and other biologics. Thus, under designated immunoassay conditions, the specified binding molecules bind preferentially to a particular peptide and do not bind in a significant amount to other proteins present in the sample. Specific binding to a polypeptide or other target under such conditions requires a binding molecule that is selected for its specificity for a particular protein. A variety of immunoassay formats may be used to select antibodies specifically immunoreactive with a particular protein or other target. For example, solid-phase ELISA immunoassays are routinely used to select monoclonal antibodies specifically immunoreactive with a protein. See, Harlow and Lane (1988) Antibodies, A Laboratory Manual, Cold Spring Harbor Publications, New York, for a description of immunoassay formats and conditions that can be used to determine specific immunoreactivity. Typically a specific reaction will be at least twice background signal or noise and more typically more than 10, 20 or 100 times background.

The ligand part of any first composition (eg ligand epitope conjugate) may be, or comprise, an ankyrin protein or a part thereof, such as comprising an ankyrin domain. The ANK domain is typically a 33-residue motif containing two antiparallel α-helices connected by a short loop.

The diseased cell is a cell that does not have a wild type phenotype, and is associated with a disease phenotype. In one aspect the diseased cell is a cancer cell. In one aspect the target cell has an antigen which is over-expressed, eg, 2, 3, 4, 5, 10 fold over expressed or more, such as a tumour associated antigen or other antigen.

The epitope part of the first composition, (eg ligand-epitope conjugate) may be a polysaccharide epitope, peptide epitope, lipid epitope or any combination thereof. The epitope comprised as a part of the first composition, (eg conjugate) may include natural or synthetic non-self sequences. In one aspect the epitope may be capable of eliciting a cytotoxic T cell response.

For the avoidance of doubt, the epitope within the epitope part is functionally only required to perform the function of an epitope, such as a T cell epitope, on processing and presentation, and may not be specifically presented or act as an epitope in the form of the first composition (e.g. ligand epitope conjugate), although this is also possible. In one aspect the epitope of the first composition (e.g. ligand-epitope conjugate does not act as an epitope in the context of the first composition (e.g. the conjugate).

As mentioned above the epitope may form a part of the ligand component, for example where the ligand is a non-self antigen, such that there is no need to take any action to conjugate the ligand and epitope together, but they pre-exist as a conjugate.

The epitope part may comprise a viral or bacterial epitope, such as an epitope selected from the group consisting of malaria epitopes, rabies epitopes, yellow fever epitopes, Influenza virus epitopes, CMV epitopes, EBV epitopes, VZV epitopes, HCV epitopes, Measles virus epitopes, mumps epitopes, Rubella virus epitopes, HPV epitopes, HBV epitopes or an epitope from an antigen not associated with disease, such as ovalbumin epitopes or a synthetic epitope. In one aspect the epitope comprises an antigen which is found in a vaccine approved by the FDA or EMEA.

The epitope part may comprise one or more epitope for which a T cell immune response already exists in the organism. For example the epitope may be, or may be part of, an antigen delivered as a part of a childhood vaccination.

The epitope may be flanked by sequences that are known to assist in the processing and/or presentation of the epitope by a cell, for example see J Gen Virol. 2004 March; 85(Pt 3):563-72, Influence of flanking sequences on presentation efficiency of a CD8+ cytotoxic T-cell epitope delivered by parvovirus-like particles.

In one aspect the first composition (e.g. ligand epitope conjugate) comprises an influenza epitope in the form of a fusion protein with an antibody heavy or light chain, or part thereof, such as a heavy chain, or part thereof, the antibody sequence or part thereof retaining binding activity for the desired target.

The fusion may be made at the C terminus of the antibody chain.

The epitope fused to the antibody chain, or part thereof, may be attached via a linker sequence, which may be flexible, such as the sequence GGSGS

The influenza protein may be all or part of the NP protein, such as:

[GGSGS]NLNDTTYQRTRALVRTGMDPRMSSLMQGSTLPRRSGAAGAAVKGIGTMVMELIRMIKRGIN
DRNFWRGENGRKTRSAYERMANILKGKFQTAAQRAMMDQVRESRNPGNAEIEDLIFLARSALILRGS
VAHK

An alternative second shorter amino acid sequence from the Influenza Nucleoprotein is SNLNDTTYQRTRALVRTGMDP.

Alternatively an ovalbumin sequence may be used, such as the sequence EQLESIINFEKLTEW, suitably fused to the C terminus of an antibody chain, or a part of an antibody chain.

An alternative second shorter amino acid sequence from the ovalbumin is SIINFEKL.

The invention also relates to:

A first composition as disclosed herein, wherein the epitope is capable of being presented on the surface of the target cell in the context of an MHC molecule.

A first composition as disclosed herein, wherein the epitope is capable of stimulating epitope-specific T cells when presented in the context of an MHC molecule.

A first composition as disclosed herein, wherein the epitope is capable of eliciting a T cell response when presented in the context of an MHC molecule.

The MHC molecule may be an MHC class 1 or 2 molecule.

The first composition (e.g. ligand epitope conjugate) of the invention is used in a method of preventing or treating disease, suitably by administering to an individual a first composition as defined herein, and an immunogenic composition, the immunogenic composition comprising an epitope which is identical to a non-self epitope of the first composition, or comprising an epitope which elicits an immune response which is cross-reactive with the non-self epitope of the ligand-epitope conjugate.

The immunogenic composition may be referred to as an immunogenic second composition herein, to more clearly distinguish the first composition from second compositions. For the avoidance of doubt the first and second compositions may both be immunogenic.

The invention also relates to a first composition (e.g. ligand epitope conjugate) as disclosed herein for use in the treatment of disease, wherein the first composition is used in conjunction together with a immunogenic second composition, the immunogenic second composition comprising an epitope which is identical to a non-self epitope of the first composition, or comprising an epitope which elicits an immune response which is cross-reactive with the non-self epitope of the first composition.

The invention also relates to a first composition (eg a ligand-epitope conjugate) as disclosed herein for use in the preparation of a medicament for the treatment of disease, wherein the first composition is used in conjunction together with a immunogenic second composition, the immunogenic second composition comprising an epitope which is identical to a non-self epitope of the first composition, or comprising an epitope which elicits an immune response which is cross-reactive with the non-self epitope of the first composition.

By cross reactive is meant that the immune response generated to non-self epitope of the immunogenic second composition is capable of recognising the presented non-self antigen of the first composition eg ligand-epitope conjugate.

An immunogenic second composition which shares such an epitope or has such a cross reactive epitope with the first composition (eg conjugate) may also be described herein as a ‘cognated’ or ‘cognate’ with a first composition (eg is a cognated immunoconjugate), that is, such an immunogenic second composition is suitable for use with a particular first composition eg conjugate.

In one aspect the immune response generated to the immunogenic second composition generates T cells specifically reactive with the presented epitope of the first composition eg conjugate.

The invention also relates to a use or method as disclosed herein wherein the immunogenic second composition is delivered: before the first composition according to any preceding claim, or after the first composition according to any preceding claim, or;

delivered concomitantly, or substantially concomitantly, such as on the same day as the first composition.

In one aspect the immunogenic composition is delivered before the first composition, such that the immune response of the individual is ready to respond to any presented non-self epitope as soon as that non-self epitope or mimetope thereof is delivered as a part of the first composition and presented by cells.

In one aspect the first composition is used in an individual with pre-existing immunity to the epitope. In such an aspect there may be no need to deliver the first composition with an immunogenic second composition, because the individual is immunologically prepared to respond to the presentation of the non-self epitope. Thus in this aspect the first composition is used without an immunogenic second composition as described herein.

The immunogenic second composition used to generate an immune response to the presented epitope may be supplemented or replaced by a passive approach to providing immunotherapy, for example by providing an agent specific to the presented epitope, such as an antibody specific to the presented epitope, or by providing a T cell population specific for the presented epitope, or by providing T cell receptors for the presented epitope in a form suitable for delivery to the body, such as soluble T cell receptors (see Molloy et al Current Opinion in Pharmacology 2005, 5:438-443 and Immunocore, http://www.immunocore.com/). In one aspect the agent is cytotoxic for the diseased cell, or capable of directing a cytotoxic response to the diseased cell. Any passive approach may be used before, after, concomitantly, or substantially concomitantly, with an immunogenic second composition as described herein which is cognated with the first composition eg ligand epitope conjugate.

In one aspect the immunogenic second composition is designed to be universal, and applicable for the treatment of different diseases. The immunogenic second composition is suitably an inducer of a CD8 and CD4 T cell immune response as well as being a non-self immunogen. The epitope in the first composition and immunogenic second composition may share a common sequence, and/or may be the same and/or derived from the same antigen. The epitope in the first composition and immunogen comprised within the immunogenic second composition may be derived from an infectious agent, such as any of those described herein in respect of ligands. The immunogenic second composition can be derived from an infectious agent such as Influenza Nucleoprotein or from other non-self proteins like for example ovalbumin. Live attenuated pathogens are considered good inducers of CD8 and CD4 immune responses and immunogens based on live attenuated viruses form an embodiment of the invention, including live attenuated measles, mumps, rubella, chicken pox, yellow fever and rabies viruses, optionally in the form of an FDA or EMEA approved vaccine.

An immunogenic second composition can comprise an entire antigenic protein or portions of it containing immunogenic epitopes. An epitope refers to a portion of an antigen capable of stimulating an immune response.

The immunogenic second composition may be a vaccine, suitably approved for use in a human population.

The immunogenic second composition may comprise an immunogen alone, or additional components such as a pharmaceutically acceptable carrier or additive.

The immunogenic second composition can comprise a protein or part thereof, including recombinant proteins, or may comprise nucleic acid encoding a protein, for example as DNA or RNA.

For immunogenic second compositions based on nucleic acid the nucleic acid may be delivered in the form of a bacterial plasmid vector into which is inserted a promoter, the gene of interest which encodes an antigenic peptide and a polyadenylation/transcriptional termination sequence. The gene of interest may encode a complete protein or simply an antigenic peptide sequence against which it is intended to raise an immune response. The plasmid can be grown in bacteria, such as for example E. coli and then isolated and prepared in an appropriate medium, depending upon the intended route of administration, before being administered to the host. Following administration the plasmid is taken up by cells of the host, or delivered directly into the host cells, where the encoded peptide is produced. The plasmid vector will preferably be made without an origin of replication functional in eukaryotic cells, in order to prevent plasmid replication in the mammalian host and integration within chromosomal DNA of the animal concerned. Helpful background information in relation to DNA vaccination is provided in Donnelly et al “DNA vaccines” Ann. Rev Immunol. 1997 15: 617-648, the disclosure of which is included herein in its entirety by way of reference. Nucleic acid may be delivered using gold beads as a carrier.

The nucleic acid may also be delivered using a viral vector such as Adenovirus or MVA. The adenovirus may be a replication defective adenovirus vector, such as a replication deficient chimpanzee adenovirus.

These means to deliver immunogenic second compositions based on recombinant viruses are currently being tested in clinic and hold promise to be even stronger inducer of CD8 and CD4 immune responses than live attenuated approaches. Adenoviral vectors and MVA coding for HCV and malaria antigens have been proven to induce very potent CD8 and CD4 immune response especially when used in a heterologous prime-boost approach. Many other non-self protein can be used in such an approach. In particular for a therapeutic approach the nucleic acid can code for antigenic parts, or all of, CMV, EBV or Influenza antigens. These viruses are known to induce a memory type CD8 response in the human population. Boosting of pre-existing T cells against a pathogen commonly present in a human population, such as one of these three pathogens, is an option for therapeutic use.

Prime-boost approaches may be used for delivery of an immunogenic composition, in which a protein or fragment thereof is delivered to an individual, followed by a nucleic acid encoding said protein or fragment thereof. Alternatively the nucleic acid may be delivered before the protein or fragment thereof. The components may each be delivered one or multiple times.

The immunogenic second composition can be used in combination with adjuvants to augment the immune response to the desired antigen and to induce long lasting protective and therapeutic immunity. An adjuvant generally refers to a component in a vaccine or immunogenic composition that increase the specific immune response to the antigen (see, e.g., Edelman, AIDS Res. Hum Retroviruses 8:1409-1411 (1992)).

Adjuvants induce immune responses of the Th1-type and Th-2 type response. Th1-type cytokines (e.g., IFN-γ, IL-2, and IL-12) tend to favour the induction of cell-mediated immune response to an administered antigen, while Th-2 type cytokines (e.g., IL-4, IL-5, Il-6, IL-10) tend to favour the induction of humoral immune responses. Adjuvants capable of preferential stimulation of a Th-1 cell-mediated immune response are described in WO 94/00153 and WO 95/17209.

Suitable adjuvants may comprise saponins such as QS21, lipid A derivatives such as MPL or 3D-MPL, oil in water emulsions such as MF59 and AS03, aluminium salts such as aluminium hydroxide and aluminium phosphate, CpG oligonucleotides and mixtures thereof. Synthetic adjuvants may also be used such as AGPs (Curr Top Med Chem. 2008; 8(2):64-79, Synthetic TLR4-active glycolipids as vaccine adjuvants and stand-alone immunotherapeutics, Johnson DA). Adjuvants may be TLR agonists, such as TLR 2, TLR3, TLR4, TLR8 and TLR9 agonists. Combinations include GSK's adjuvant combination of AS01 having QS21 and 3D MPL.

A review of some adjuvants is disclosed in “Expert Rev Vaccines. 2007 October; 6(5):723-39. GlaxoSmithKline Adjuvant Systems in vaccines: concepts, achievements and perspectives. Garcon N et al. Other adjuvants are disclosed in VACCINE ADJUVANTS, Infectious Disease, 2006, 235-255, DOI: 10.1007/978-1-59259-970-7_{—}12.

Preferred adjuvants for use in eliciting a predominantly Th1-type response include, for example, a combination of monophosphoryl lipid A (MPL®) preferably 3-O-deacylated monophosphoryl lipid A (3D-MPL®), optionally with an aluminium salt (see, for example, Ribi, et al., 1986, Immunology and Immunopharmacology of Bacterial Endotoxins, Plenum Publ. Corp., NY, pp. 407-419; GB 2122204B; GB 222021 1; and U.S. Pat. No. 4,912,094). A preferred form of 3D-MPL® is in the form of an emulsion having a small particle size less than 0.2 mm in diameter, and its method of manufacture is disclosed in WO 94/21292. Aqueous formulations comprising monophosphoryl lipid A and a surfactant have been described in WO 98/43670. Exemplified preferred adjuvants include AS01 B (MPL® and QS21 in a liposome formulation), 3D-MPL® and QS21 in a liposome formulation, AS02A (MPL® and QS21 and an oil in water emulsion), 3D-MPL® and QS21 and an oil in water emulsion, and AS15, available from GlaxoSmithKline. MPL® adjuvants are available from GlaxoSmithKline (see U.S. Pat. Nos. 4,436,727; 4,877,611; 4,866,034 and 4,912,094).

CpG-containing oligonucleotides (in which the CpG dinucleotide is unmethylated) also induce a predominantly Th1 response. CpG is an abbreviation for cytosine-guanosine dinucleotide motifs present in DNA. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). CpG when formulated into immunogenic compositions, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8). CpG is known in the art as being an adjuvant that can be administered by both systemic and mucosal routes (WO 96/02555, EP 468520, Davis et al., J. Immunol, 1998, 160(2):870-876; McCluskie and Davis, J. Immunol., 1998, 161 (9):4463-6).

Another preferred adjuvant is a saponin or saponin mimetics or derivatives, such as Quil A, preferably QS21 (Aquila Biopharmaceuticals Inc., Framingham, Mass.), which may be used alone or in combination with other adjuvants. For example, an enhanced system involves the combination of a monophosphoryl lipid A (MPL®) and saponin derivative, such as the combination of QS21 and 3D-MPL® as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL® and tocopherol in an oil-in-water emulsion is described in WO 95/17210. Additional saponin adjuvants of use in the present invention include QS7 (described in WO 96/33739 and WO 96/1171 1) and QS17 (described in U.S. Pat. No. 5,057,540 and EP 0 362 279 B1).

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOM's. Furthermore, the saponins may be formulated together with a polyoxyethylene ether or ester, in either a non-particulate solution or suspension, or in a particulate structure such as a paucilamelar liposome or ISCOW). The saponins may also be formulated with excipients such as CARBOPOL® to increase viscosity, or may be formulated in a dry powder form with a powder excipient such as lactose.

In one embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol containing liposomes, as described in WO 96/33739. Other suitable formulations comprise an oil-in-water emulsion and tocopherol. Another suitable adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Suitably the formulation additionally comprises an oil in water emulsion and tocopherol. Other suitable adjuvants include MONTANIDE® ISA 720 (Seppic, France), SAF (Chiron, Calif., United States), ISCOMS® (CSL), MF-59 (Chiron), the AS series of adjuvants (GlaxoSmithKline, Rixensart, Belgium), Detox (Corixa), RC-529 (Corixa) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1. Corixa Corporation is now part of GlaxoSmithKline.

Other suitable adjuvants include adjuvant molecules of the general formula (I):

HO(CH₂CH₂O)n-A-R wherein, n is 1-50, A is a bond or —C(O)—, R is C1-50 alkyl or Phenyl C1-50 alkyl. A further adjuvant of interest is shiga toxin b chain, used for example as described in W02005/1 12991. One embodiment of the present invention consists of an immunogenic composition comprising a polyoxyethylene ether of general formula (I), wherein n is between 1 and 50, preferably 4-24, most preferably 9; the R component is C1-50, preferably C4-C20 alkyl and most preferably C12 alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12th edition: entry 7717). These adjuvant molecules are described in WO 99/52549.

In one aspect the adjuvant is effective in promoting a cytotoxic T cell response, such that the target cell is killed.

In the case of a nucleic acid the adjuvant can be a sequence encoded in the nucleic acid component itself.

Within the immunogenic compositions provided herein, the adjuvant composition is preferably designed to induce an immune response predominantly of the Th1 type. Following application of a immunogenic composition as provided herein, a patient will typically support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Janeway, et al., Immunobiology, 5th Edition, 2001.

The present invention also relates to certain specific immunogenic compositions, and uses thereof, such as an immunogenic composition comprising a non-self epitope for use in the treatment or prevention of disease, such as non-infectious diseases and cancer. The invention also relates to an immunogenic composition comprising a non-self epitope which is not a tumour associated antigen for the treatment or prevention of cancer. In one embodiment the composition does not include a tumour associated antigen or epitope. In one aspect the immunogenic compositions may be used in an individual who has cells which present the epitope.

The invention also relates to an immunogenic composition comprising an antigen or epitope which is not associated with any human or animal disease, optionally in combination with a pharmaceutically acceptable excipient. In one embodiment the antigen is not a self-antigen. The antigen may be a synthetic antigen, for example. The antigen or epitope may be a non-human, non-bacterial non-viral epitope or antigen. In this sense an immunogenic composition may be generated which can be used universally in all people, whatever disease it is desired to treat. The immunogenic composition is designed to generate an immune response to a non-self antigen whereas the disease specificity is achieved by the ligand part of the first composition, which can be selected to target different disease cells.

The immunogenic second composition is suitable for use in a human and may comprise a pharmaceutically acceptable adjuvant or carrier or excipient.

Processing and presentation by the cell may be assessed empirically by providing a cell with a first composition such as a ligand epitope conjugate under conditions suitable to allow the binding of the ligand part of the first composition to the target, and assessing the reactivity of the cell with a suitable T cell population which has been generated against a cognate immunogenic second composition.

It will be appreciated that the methods and compositions of the invention may be for the treatment of any disease associated with abnormal expression of a self-antigen. The methods and compositions of the invention may be for the treatment of cancer, which may be solid tumour or hematologic.

The invention also relates to a kit of parts comprising
a) a first composition eg a ligand-epitope conjugate as defined herein and
b) a immunogenic second composition as defined herein

The range of concentrations for the first composition can vary according to product binding properties and in vivo affinities and half life, for example Herceptin is dosed at 2-4 mg/kg body weight.

The compositions, compounds and conjugates of the present invention may be administered at a daily dosage of from about 0.005 mg to about 100 mg per kg of animal body weight, for example given as a single daily dose or in divided doses two to six times a day, or in sustained release form. The total daily dosage is from about 0.01 mg to about 2000 mg, preferably from about 0.1 mg to about 20 mg per kg of body weight. This dosage regimen may be adjusted to provide the optimal therapeutic response. The compounds may be administered on a regimen of 1 to 4 times per day, preferably once or twice per day.

The dose of the first composition eg conjugate and immunogenic second composition may be different, and each may be delivered by the same or a different administration route.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The first composition eg conjugate and immunogenic second composition may be administered to a patient in need thereof by any conventional route of administration, including, but not limited to, intravenous, intramuscular, oral, subcutaneous, intradermal, and parenteral delivery.

Dosing of the immunogenic second composition depends on the materials within the composition. For example, adenovirus based vaccines may be administered at doses in the range of 10⁸-10¹¹ viral particles, while MVA based vaccines may be administered at doses in the range of 10⁷-5×10⁸ plaque forming units. If a vaccine already exists corresponding to the first composition eg a conjugated epitope (for instance influenza) then it may be administered at the recommended concentration/dose.

The amount of antigen in each dose of first composition or immunogenic second composition is selected as an amount which induces an immunoprotective response without significant, adverse side effects in typical subjects. Such amount will vary depending on which specific antigens are employed. Generally it is expected that each dose will comprise 1-1000 μg of total antigen, preferably 2-200 μg. An optimal amount for a particular first composition or immunogenic second composition can be ascertained by standard studies involving observation of antibody titres and other responses in subjects. Following an initial vaccination, subjects may receive one or more booster doses, for example after 2 and 6 months.

Immunogenic compositions may be prepared in accordance with vaccine preparations, with Vaccine preparation being generally described in New Trends and Developments in Vaccines, edited by Voller et al., University Park Press, Baltimore, Md. U.S.A. 1978.

Certain aspects of the invention include:

A ligand-epitope conjugate comprising
a ligand part being able to specifically bind to a target, the target being on a diseased cell of an organism, and
an epitope part having a non-self epitope not naturally being encoded by the organism, wherein
the target is internalized after forming a complex with the conjugate and the non-self epitope is presented on the cell surface of said diseased cell of an organism after internalization.

A ligand-epitope conjugate as disclosed herein, wherein the ligand part of said conjugate is coupled to the epitope part by chemical cross linking.

A ligand-epitope conjugate as disclosed herein, wherein said conjugate is produced by expressing a fusion protein comprising the ligand part and the epitope part.

A ligand-epitope conjugate as disclosed herein wherein the target is a receptor or antigen.

A ligand-epitope conjugate as disclosed herein wherein the target is specific to a tumour cell, or has an expression which is different from a wild type cell, such as being over-expressed.

A ligand-epitope conjugate as disclosed herein, wherein said target is a receptor tyrosine kinase.

A ligand-epitope conjugate as disclosed herein, wherein the receptor tyrosine kinase is selected from the group consisting of:

The ErbB or Epidermal Growth Factor (EGF) family of receptor tyrosine kinases (RTKs) such as: EGF R/ErbB1/HER1, ErbB2/Neu/HER2, ErbB3/HER3, and ErbB4/HER4;
The EPH family of receptor tyrosine kinases (EPHA1, EPHA2, EPHA3, EPHA4, EPHA5, EPHA6, EPHA7, EPHA8, EPHA9, EPHA10; EPHB1, EPHB2, EPHB3, EPHB4, EPHB5, EPHB6)
The c/MET family (Met and RON);
The VEGF family including VEGFR-1 (Flt-1) VEGFR-2 (KDR/Flk-1) and VEGFR-3(Flt-4)

A ligand-epitope conjugate as disclosed herein, wherein the ligand part of said conjugate is an antibody or an antigen-binding fragment of an antibody.

A ligand-epitope conjugate as disclosed herein, wherein the target is a receptor and the ligand part of said conjugate is a ligand for the receptor.

A ligand-epitope conjugate as disclosed herein wherein the ligand is selected from:

a) the natural ligand or receptor binding fragments thereof;
b) mimics of the ligand such as peptides or synthetic molecules;
c) non-natural ligands and their mimics

A ligand-epitope conjugate as disclosed herein wherein the target for the ligand is a receptor tyrosine kinase.

A ligand-epitope conjugate as disclosed herein wherein the ligand is selected from EGF, TGF-a, HB-EGF, amphiregulin, betacellulin, epigen, epiregulin, neuregulin 1, 2, 3, 4, EFNA1, 2, 3, 4, 5, EFNB1, 2, 3, Hepatocyte growth factor (HGF), macrophage stimulating protein (MSP), VEGF-A, VEGF-C and VEGF-D;

A ligand-epitope conjugate as disclosed herein, wherein the diseased cell is a cancer cell.

A ligand-epitope conjugate as disclosed herein, wherein the epitope is a polysaccharide epitope, peptide epitope, lipid epitope or any combination thereof.

A ligand-epitope conjugate as disclosed herein, wherein the epitope is capable of eliciting a cytotoxic T cell response when presented with an MHC molecule.

A ligand-epitope conjugate as disclosed herein, wherein the epitope forms a part of an antigen against which there is routine childhood vaccination.

A ligand-epitope conjugate as disclosed herein, wherein the epitope forms a part of the ligand component, and wherein the ligand is a non-self antigen.

A ligand-epitope conjugate as disclosed herein, wherein said epitope part comprises one or more epitopes selected from the group consisting of Influenza virus epitopes, CMV epitopes, EBV epitopes, VZV epitopes, HCV epitopes, HBV epitopes, Measles virus epitopes, Rubella virus epitopes, rabies virus epitopes, yellow fever virus epitopes, epitopes from an antigen not associated with disease such as ovalbumin; and synthetic epitopes.

A ligand-epitope conjugate as disclosed herein, wherein said epitope part comprises an epitope against which for which a T cell immune response already exists in the organism.

A ligand-epitope conjugate as disclosed herein for use in the treatment of disease, wherein the conjugate is used in conjunction together with an immunogenic composition comprising an epitope which is identical to a non-self epitope of the ligand-epitope conjugate, or comprising an epitope which elicits an immune response which is cross-reactive with the non-self epitope of the ligand-epitope conjugate.

A ligand-epitope conjugate as disclosed herein for use in preparation of a medicament for the treatment of disease, wherein the conjugate is used in conjunction together with an immunogenic composition comprising an epitope which is identical to a non-self epitope of the ligand-epitope conjugate, or comprising an epitope which elicits an immune response which is cross-reactive with the non-self epitope of the ligand-epitope conjugate.

A method of treating disease by administering to an organism

a) a ligand-epitope conjugate as disclosed herein and
b) an immunogenic composition comprising an epitope which is identical to a non-self epitope of the ligand-epitope conjugate, or comprising an epitope which elicits an immune response which is cross-reactive with the non-self epitope of the ligand-epitope conjugate.

Conjugate or method as disclosed herein wherein the immunogenic composition is delivered:

before the ligand-epitope conjugate according to any preceding claim, or
after the ligand epitope conjugate according to any preceding claim, or
concomitantly, or substantially concomitantly, such as on the same day, as the immunogenic composition.

A conjugate or method as disclosed herein used in an individual with pre-existing immune response to the epitope.

A ligand-epitope conjugate as disclosed herein wherein the conjugate is used in an individual with a pre-existing immune response to the epitope and without use of a cognate immunogenic composition.

A method of prevention or treatment of disease, the method comprising delivery of a ligand-epitope conjugate as disclosed herein to an individual with a pre-existing immune response to the epitope, and without use of a cognate immunogenic composition.

A ligand-epitope conjugate as disclosed herein for use in the treatment of disease, or in the preparation of a medicament for the treatment of disease, wherein the conjugate is used in conjunction with an agent that is specific for the presented non-self epitope.

A method of medical treatment comprising delivering to an individual in need thereof an effective amount of a ligand-epitope conjugate as disclosed herein in conjunction with an agent that is specific for the presented non-self epitope.

A ligand-epitope conjugate or method as disclosed herein wherein the agent is an antibody or fragment thereof which binds to the same target as the whole antibody, or a population of cytotoxic T cells.

A ligand-epitope conjugate or method as disclosed herein, wherein the conjugate and agent are used together with an immunogenic composition comprising an epitope which is identical to a non-self epitope of the ligand-epitope conjugate, or comprising an epitope which elicits an immune response which is cross-reactive with the non-self epitope of the ligand-epitope conjugate

A immunogenic composition comprising a non-self antigen for use in the treatment or prevention of non-infectious disease.

A immunogenic composition as disclosed herein comprising an antigen which is not a tumour associated antigen for the treatment or prevention of cancer.

A immunogenic composition as disclosed herein wherein the immunogenic composition does not including a tumour associated antigen.

A pharmaceutically acceptable immunogenic composition comprising a non-self antigen which is not associated with any disease.

A method or conjugate or immunogenic composition as disclosed herein wherein the immunogenic composition additionally comprises an adjuvant.

A method or use or immunogenic composition as disclosed herein wherein the adjuvant is effective in promoting a cytotoxic T cell response

A method or use as disclosed herein wherein the use is for a disease associated with abnormal expression of a self antigen

A method or use as disclosed herein wherein the use is for the treatment of solid tumour or hematologic cancer.

A kit of parts comprising

a) a ligand-epitope conjugate as disclosed herein; and
b) a immunogenic composition as disclosed herein.

All references or patent applications cited within this patent specification are incorporated by reference herein.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, where relevant, the term “about” is used to indicate that a value includes the inherent variation of error for the device, the method being employed to determine the value, or the variation that exists among the study subjects.

Any feature of the invention is disclosed as a discreet combination with any other feature or features of the invention, unless otherwise apparent from the context.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled person will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Kazansky, D. B., Intrathymic selection: new insight into tumor immunology. Adv Exp Med Biol, 2007. 601: p. 133-44.
2. Storkus, W. J., et al., Improving immunotherapy by conditionally enhancing MHC class I presentation of tumor antigen-derived Peptide epitopes. Crit Rev Immunol, 2007. 27(5): p. 485-93.
3. Park, S., et al., The therapeutic effect of anti-HER2/neu antibody depends on both innate and adaptive immunity. Cancer Cell, 2010. 18(2): p. 160-70.
4. Kono, K., et al., Trastuzumab (Herceptin) enhances class I-restricted antigen presentation recognized by HER-2/neu-specific T cytotoxic lymphocytes. Clin Cancer Res, 2004. 10(7): p. 2538-44.
5. Milano, F., et al., Trastuzumab mediated T-cell response against HER-2/neu overexpressing esophageal adenocarcinoma depends on intact antigen processing machinery. PLoS One, 2010. 5(8): p. e12424.
6. zum Buschenfelde, C. M., et al., Antihuman epidermal growth factor receptor 2 (HER2) monoclonal antibody trastuzumab enhances cytolytic activity of class I-restricted HER2-specific T lymphocytes against HER2-overexpressing tumor cells. Cancer Res, 2002. 62(8): p. 2244-7.
7. Mittendorf, E. A., et al., Investigating the combination of trastuzumab and HER2/neu peptide vaccines for the treatment of breast cancer. Ann Surg Oncol, 2006. 13(8): p. 1085-98.
8. Wesa, A. K., et al., Enhancement in specific CD8+ T cell recognition of EphA2+ tumors in vitro and in vivo after treatment with ligand agonists. J Immunol, 2008. 181(11): p. 7721-7.
9. clinicaltrials.gov, A Study of CDX-1307, in Patients With Incurable Breast, Colorectal, Pancreatic, Ovarian or Bladder Cancer (CDX 1307-01). Sponsor: Celldex Therapeutics 2010. Status:completed.
10. clinicaltrials.gov, A Study of CDX-1401 in Patients With Malignancies Known to Express NY-ESO-1. Sponsor: Celldex Therapeutics 2010 status: recruiting
11. clinicaltrials.gov, Phase I Study of CDX-1307, hCG-B Vaccine, for Patients With Incurable, Locally Advanced or Metastatic Breast, Colorectal, Pancreatic, Bladder or Ovarian Cancer (CDX1307-02). Sponsor: Celldex Therapeutics 2010. Status: Completed

In order that this invention may be better understood, the following examples are set forth. These examples are for purposes of illustration only, and are not to be construed as limiting the scope of the invention in any manner.

### EXAMPLES

The concept will be validated by the following experiment.

A ligand epitope conjugate will comprise an antibody or a ligand well characterized for its internalization properties, linked to a non-self peptide antigenic sequence.

An immunogenic composition will comprise the non-self sequence peptide in a well known and validated immunogenic format.

### In Vitro Experiments:

Preliminary screening of non-self ligand can be performed in vitro:

A direct measure of the non-self peptide loaded on MHC molecules by tumor cells can be obtained by using specific reagents (soluble TCR or TCR mimic antibodies) recognizing the complex formed by the non-self peptide and the MHC (e.g. where the non-self epitope is comprised within an ovalbumin polypeptide, and experiments are performed in C57Bl/6 mice then antibodies may recognise an ova peptide in the context of murine C57Bl/6 MHC). Syngenic tumor cells will be incubated with a ligand-epitope conjugate and tested for the presence of the peptide complexes with MHC on the cell surface.

The loading of the non self (NS) peptide on a tumor cell surface can also be measured indirectly by monitoring the activation status of T cells specifically recognizing the non-self peptide in the appropriate MHC context. In vitro co-incubation of a series of syngenic tumor cells with T cells specific for the non-self peptide will result in activation of the specific T cells functions only in the presence of the ligand epitope conjugate and following appropriate processing and presentation.

T cells specific for the NS can be obtained by immunizing syngenic mice with immunogenic compositions as described above.

Several assays can be used to measure the T cell activation.

Released IFN-γ or other cytokines can be measured by enzyme-linked immunosorbent assay (ELISA) or other assays after incubation of NS specific T cells with target tumor cells in the presence of the ligand epitope conjugate

Cytotoxicity against target tumor cells can be measured by a classical 51Cr release assay after co-incubation of activated T cells and 51Cr labelled target tumor cells in the presence of the ligand epitope conjugate

T cell proliferation can be monitored after incubation of non-self specific T cells with ligand epitope treated syngenic tumor cells

### In Vivo Experiments:

The approach can be finally tested in vivo by using validated mice models for studying immunotherapy

Activity of the two components approach can be tested on spontaneous (i.e. BALB-neuT) and engrafted mice tumors. An immunogenic composition will be delivered to mice either in a prophylactic or in a therapeutic manner in a well known and validated immunogenic format. The conjugate (e.g. an antibody reactive against the rat-neu or an anti-EPHA2 antibody) will be then injected and the growth of spontaneous mammary tumours monitored. To test the concept on engrafted tumours several murine tumour cell lines are available. In the engrafted models mice will be vaccinated either in a prophylactic or in a therapeutic manner with the immunogenic composition and then treated with the ligand epitope conjugate (i.e. an antibody reactive against the rat-neu or an anti-EPHA2 antibody).

The efficacy of this approach can be also tested on human tumours engrafted in nude mice. The ligand epitope conjugate can comprise an antibody or a ligand against human tumor receptors able to induce internalization (e.g. against human HER-2, EphA2 or others). In this experimental setting, T cell clones recognizing the non-self peptide in a specific HLA context (i.e. HLA-A2) will be passively transferred in tumor engrafted mice and served as surrogate for the NS-vaccine.

### Example 1

Binding to cancer cells of anti-EphA2 and anti-ErbB2 monoclonal antibodies fused to an ovalbumin (OVA) peptide reproducing a CD8 T cell epitope stimulates OVA-specific CD8 T cells

### Cell Lines and Antibodies

Cell lines MC38 (mouse colon cancer), C35 Luc (a mutant of the mouse Hepa-1c1c7 luciferase-expressing cell line), HCT 116 (human colon carcinoma), A549 (human lung adenocarcinoma) and HEK-293-EBNA (from human embryonic kidney), were cultured in Dulbecco's Modified Eagle Medium (DMeM) (GIBCO). The 4T1 (mouse breast cancer), SKBR3 (mouse breast cancer) and TUBO cell lines (BALB-neuT mouse-derived mammary lobular carcinoma), were grown in RPMI medium (GIBCO). The media were supplemented with 10% heat-inactivated foetal bovine serum (FBS), 20% FBS for TUBO cells, 2 mM L-glutamine, 100 unit/mL penicillin, 100 μg/ml streptomycin.

In addition to DMEM complete medium, 400μ/ml and 250 μg/mL of G418 was used for C35 Luc and HEK-293-EBNA, respectively and 0.1 nnM of non-essential amino acids (NEAA from GIBCO) was added for HEK-293-EBNA.

EBNA-293/EphA2 cells (a 293 cell line stably transfected with the human EphA2 gene under the control of an inducible promoter) were cultured in complete DMEM with G418 (250 μg/mL) puromicin 0.5μ/ml and hygromycin 50 μg/ml.

All cell lines were cultured at 37° C. in 5% CO2 atmosphere.

The antibodies used were:

rabbit polyclonal anti-EphA2 (Santa Cruz Biotechnology);
rabbit polyclonal anti-actin (Santa Cruz Biotechnology);
goat anti-human IgG1 horseradish peroxidase-conjugated; (Promega);
anti-mouse IgG horseradish peroxidase-conjugated (Techno Scientific);
anti-rabbit IgG horseradish peroxidase-conjugated (Sigma);
25 D1.16 (PE-conjugated) specific for the H-2Kb-SIINFEKL peptide of ovalbumin in the context of major histocompatibility class I (eBioscience);
monoclonal anti-ErbB2 (Cell Signaling Tecnology);
anti-EphA2 antibodies: nnAb15 and the resulting OVA peptide fusion antibodies, nnAb15-OVApep2 and nnAb15-OVApep1, [whose heavy chain is fused to amino-acid long peptide residues 257-264 (SIINFEKL) or residues 253-267 (EQLESIINFEKLTEW) from the chicken ovalbumin] were prepared as described below.
Anti-ErbB2 (Herceptin) and the resulting OVA peptide fusion antibodies, Herceptin-OVAPep2 [whose heavy chain is fused to amino-acid long peptide residues 257-264 (SIINFEKL) from the chicken ovalbumin] were prepared as described below.

### Plasmids and Adenoviral Vector

The plasmids used were: pEU 1.2 gateway, a 9.3 kb plasmid containing an expression cassette for the human gamma 1 heavy chain, and pEU 3.2 a 6.4 kb plasmid containing an expression cassette for the human kappa light chain.

The expression cassette includes the EF-1 alpha promoter, the SV40/poly A sequence and the secretory leader sequence: MGWSCIILFLVATATG.

These plasmids also contain the Epstein Barr origin of replication (ori P), to increase the gene copy number and the levels of transient expression from HEK 293-EBNA.

DNA fragments encoding the heavy and light variable domains VH and VL of human anti-EphA2 mAb 15 were cloned in the polylinker region of pEU 1.2 and pEU 3.2 plasmids, respectively, between the secretory leader sequence and the gamma 1 or kappa constant region sequences. The resulting plasmids were named pEU 1.2 VH15 and pEU 3.2 VL15. Plasmid pEU 1.2 VH15 was used for subcloning (SfiI/HindIII) a DNA fragment encoding peptide SIINFEKL (OVAPep2) or peptide EQLESIINFEKLTEW (OVAPep1) at the C-terminus of the Heavy chain. The genes were codon-optimized to increase their expression in human cell lines. The resulting plasmids were named pEU 1.2 VH15-OVAPep2 and pEU 1.2 VH15-OVAPep1.

DNA fragments encoding heavy and light variable domains VH and VL of human anti-ErbB2 Herceptin were cloned into the polylinker region of pEU 1.2 and pEU 3.2 plasmids respectively between the secretory leader sequence and the gamma 1 or kappa constant region sequence. The resulting plasmids were named pEU 1.2 HH and pEU 3.2 HVL. The plasmid pEU 1.2 HH was used for subcloning (BsrGI/XbaI) the DNA encoding peptide SIINFEKL (OVAPep2) at the CH3 C-terminal sequence. The gene was codon-optimized to increase their expression in human cell lines. The resulting plasmids were named pEU 1.2 HH-OVAPep2.

Recombinant human Adenovirus serotype 5 expressing a chicken ovalbumin, Ad5 OVA TAG was amplified by serial passages and purified through CsCl gradient as described previously. Briefly, 2.5×106 HEK 293 cells planted on 15 cm culture dishes were infected with Ad5OVA TAG at an MOI of 100 VP/cell. Complete cytopathic effect was observed about 2 days post infection. Cells and supernatant were harvested, freeze-thawed three times, clarified by centrifugation at 2,000 rpm for 20 min at room temperature (RT). The resulting lysate was serially passaged on HEK 293 cells to increase the titer of the rescued virus. A large prep was obtained by infection of 2 cell factories (Nunc) and purification of the cell lysate on CsCl gradient. The viruses harvested from CsCl purification were transferred in the slide cassette and dialyzed against the buffer A195 (10 mM TRIS, 10 mM Histidine, 5% sucrose, 75 mM NaCl, 1 mM MgCl2, 0.02% PS-80, 0.1 mM EDTA, 0.5% (v/v) Ethanol). To determine the number of viral particles (VP), the CsCl-purified viruses were diluted 1/100 in 0.1% sodium dodecyl sulfate-phosphate-buffered saline (PBS). As a control, buffer A195 was used. These dilutions were incubated for 10 min at 56° C. The number of VP/mL was calculated by quantitative real time reverse transcription-PCR (Taqman) using primers/probes (6-FAM-TAMRA) within the CMV sequence. Standard curves were prepared using plasmids containing the same sequence.

### DNA Plasmid Preparation

100 ng of each plasmid (pEU1.2 VH15, pEU 1.2 VH15-OVAPep2, pEU 1.2 VH15 OVAPep1, pEU3.2 VL15, pEU 1.2 HH and pEU 3.2 HVL) were transformed into E. coli strain (DH5a, Invitrogen). A single colony was picked from freshly streaked bacteria on antibiotic selective plate and inoculated in 5 ml LB medium containing ampicillin (100 μg/ml). Cells were incubated for approximately 6 hours at 37° C. with vigorous shaking. The starter culture was diluited 1:200 in 1 L of LB medium with selection and grown overnight with constant agitation.

Large amounts of plasmids, required for the transfection of eukaryotic cells, were obtained by Endotoxin free Plasmid Giga Kit (Qiagen), according to the manufacturer's instructions.

### Peptides

Synthetic peptide corresponding to the amino acid sequence 257-264 of the chicken ovalbumin (SIINFEKL), and the unrelated control peptide NS3 1480 (GAUQNEVTL) were synthesized (95% purity) by Peptide Chemistry Unit at IRBM Science Park, Italy. Peptides were reconstituted in 100% DMSO at 20 mg/mL.

Production and Purification of α-EphA2 and α-ErbB2 mAbs

HEK 293-EBNA cells were transiently co-transfected with expression plasmids encoding wild type mAbs and mAb fusions to OVA peptides using the Lipofectamine 2000 Transfection Reagent (Invitrogen, USA) according to the manufacturer's instructions.

Cells were seeded 2 days before transfection and transfected at approximately 90% confluency. Routinely, 90 μl of Lipofectamine 2000 and 60 μg of each plasmid DNA were diluted separately with the Opti-MEM I Reduced-Serum medium (Invitrogen) in a final volume of 2 ml each. After 5 min of incubation, DNA and liposomal agents were combined and incubated together for 20 min to enable liposome-DNA complex formation. During this time cells were washed once with PBS and once with Opti-MEM medium to remove serum and antibiotics. The liposome-DNA mix was added to the cells in 16 ml of the Opti-MEM medium for 6 hours at 37° C. Then the overlay was replaced with IgG production medium (CD-CHO medium without FBS) (GIBCO) and incubated at 37° C. in a 5% CO2 humidified atmosphere. Culture medium was harvested usually 10-12 days post-transfection, clarified by centrifugation (1200 rpm for 10 min) and checked for immunoglobulin (IgGs) production by 4-12% gradient SDS-PAGE followed by Coomassie Blue staining.

Whole human IgGs secreted into the medium (with an estimated IgG concentration of about 100-150 mg/L) were purified by affinity chromatography as follows: clarified culture medium (50-100 mL) was loaded at 0.5 ml/min onto a 1 mL HiTrap Protein A HP Columns (GE Healthcare) equilibrated in 20 mM sodium phosphate pH 7. Following a washing step with 6 column volumes (CV) of 20 mM sodium phosphate pH 7, bound IgGs were eluted with 6 CV of 0.1 M glycine-HCl buffer, pH 2.7. The eluted material was immediately neutralized with 1/5 volume of 1M Tris-HCl, pH 9.0.

The purity of the preparation was evaluated by 4-12% gradient SDS-PAGE under reducing condition, followed by Coomassie staining. Protein concentration was determined by BCA assay kit (PIERCE) and pooled fractions were stored until use at 80° C.

### Cell Lysates and Western Blot Analysis

MC38, 4T1, HCT 116 and TUBO cells were detached with cell dissociation solution (Sigma) and washed in PBS. Lysates were prepared by resuspending 4×10̂6 cells in 150 uL of lysis buffer [10 mM Tris-HCl (pH 7.4), 150 mM NaCl, and 0.5% NP40 containing Complete proteases inhibitor (Roche)]. After 20 min at 0° C., extracts were clarified by centrifugation at 12,000 rpm for 10 min. The protein extracts were quantified using the BCA assay kit according to the manufacturer's instructions. 50 μg of each sample were resolved by 4-12% SDSPAGE, electro-blotted onto nitrocellulose membranes (Millipore Corporation) and probed with anti-EphA2 and α-actin to verify equivalent sample loading, followed by anti-rabbit IgG HRP conjugated.

Immunoreactive proteins were visualized by phosphorimager analysis of enhanced chemiluminescence (Super Signal®West-Pico Chemiluminescent Substrate, Pierce).

ELISA Assays with EphA2 and ErbB2 Positive Cells

ELISA assays with EphA2-positive cells were carried out on MC38, C35 Luc and EBNA-293/EphA2 treated or untreated with doxycicline (50 ug/mL for 16 h).

ELISA assays with ErbB2-positive cells were carried out on SKBR3, MC38 and C35 Luc.

Cells were harvested in non-enzymatic dissociation solution, washed and transferred to U-bottom 96-well microtiter plates (2×105 cells per well). After blocking with PBS containing 6% BSA, plates were washed and purified anti-EpHA2 mAbs (nnAb15, nnAb15-OVAPep2 and nnAb15-OVAPep1) or anti-ErbB2 mAbs (Herceptin and Herceptin-OVAPep2) were added at increasing concentrations (0.2-2 μM) in ELISA buffer (PBS/BSA 3%) in triplicate wells then incubated for 2 hrs at room temperature with a blank control of PBS. After incubation, centrifugation and removal of supernatants, pelleted cells were washed twice with PBS and incubated with peroxidase-conjugated anti-human IgG1 (1/2500 in ELISA buffer) for antibody detection. After 1 h, the plates were centrifuged, washed with PBS, and bound antibodies were detected by using 3,3′,5,5-tetramethylbenzidine (TMB) as a substrate (Sigma). Binding values were determined from the absorbance at 450 nm using a microplate reader (Multilabel Counter Victor 3, Perkin Elmer) and reported as the mean of at least three determinations.

### Antigen Presentation Assays by Flow Cytometry

Presentation of the OVA257-264/H-2Kb complex by mAb-OVA fusions specific for EphA2 and ErbB2 positive cells was determined as follows.

Murine EphA2 positive and ErbB2 positive cells MC38 and C35 Luc (aplotype H-2Kb) were harvested in non-enzymatic dissociation solution, washed and transferred to 6-well microtiter plates (4×105 cells per well). The cells were treated with increasing concentration of purified wild type or OVA-antibody fusions (50 nM-4 uM) for 48 hours at 37° C. As positive control cells were treated with 2 μM of peptide SIINFEKL (OVA257-264) for 1 h at 37° C.

After incubation, cells were harvested, transferred into FACS tubes, washed with FACS buffer (PBS, containing 2 mM Hepes, BSA 2%) and centrifuged for 10 minutes at 1200 rpm.

Cells were incubated with 25 D1.16 antibody PE-conjugated, specific for the OVA257-264/Kb complex in the context of MHC class I.

After 1 h at RT and 1 hour at 37° C. with vigorous shaking, cells were washed twice with FACS buffer. Samples were resuspended in 200 μl of PBS and analyzed by flow cytometry using a Becton Dickinson calibur flow cytometer (BD Biosciences).

### Mice

C57BL/6 (H-2Kb) mice were obtained from the Charles River Laboratory. All mice were kept in temperature, air, and light-controlled (light on from 7 a.m. to 7 p.m.) conditions and received food and water ad libitum. All animals received humane care according to the criteria outlined in the Guide for the Care and Use of Laboratory Animals.

### Mice Immunization

Six-week-old female C57BL/6 (H-2b) mice were immunized with 108 vp of Ad5OVATAG in 100 μl of sterile A195 buffer (see above). In particular, mice were fully anesthetized with Tribromoethanol (Avertin) at 125-250 mg/kg IP (intraperitoneal) of body weight, and two injections were performed into the quadriceps muscle of each hind limb. 10 and 21 days after the injections, mice were euthanized, and spleens were harvested and processed for detection of T cell responses by IFN-γ ELIspot assays.

### Ex Vivo IFN-γ ELISPOT

Antigen-specific T cells secreting IFN-γ were detected using a standard enzyme-linked immunospot (ELISPOT) assay. A 96 well plate with PVDF membrane (Millipore) was coated with 100 μl/well of anti-mouse IFN-C antibody (U-Cytech) diluted to 10 μg/ml in PBS and stored overnight at 4° C. The following day, wells were blocked with 200 μl/well of R10 medium (RPMI medium 1640 with 10% heat inactivated FBS, 1M HEPES buffer diluted 1:100, 200 mM L-glutamine diluted 1:100, 100X penicillin-streptomicin solution diluted 1:100, 55 mM 2-mercaptoethanol diluted 1:1000 all from GIBCO) and incubated at 37° C. for 2 hrs.

The spleens from immunized mice were homogenized and a single cell suspension was prepared by lysing red blood cells with ACK lysis buffer (0.15 M NH4Cl, 1.0 nnM KHCO3, 0.1 M Na2EDTA, pH 7.2) (GIBCO). Cells resuspended at 8×106 cells/ml in complete R10 medium and 25 or 50 μl/well corresponding to 2×105 and 4×105/cells were added to wells in duplicate.

OVA-specific CD8 T cells from mouse splenocytes were stimulated with:

EphA2 positive cells C35 Luc (2×105) previously treated or not treated with increasing concentrations (50 nM-4 μM) of purified anti-EphA2 mAbs (nnAb15, nnAb15-OVAPep2 and nnAb15-OVAPep1) for 48 hours at 37° C., or pulsed with SIINFEKL peptide (2 μM) for 1 h a at 37° C. as positive control;
ErbB2 positive cells C35 Luc (2×105) previously treated or not treated with purified anti-ErbB2 MAbs (Herceptin and Herceptin-OVAPep2) 4 μM for 48 hours at 37° C., or pulsed with SIINFEKL peptide (2 μM) for 1 h a at 37° C. as positive control;
Fifty microliters per well of SIINFEKL peptide or unrelated control peptide (final concentration 50 ng/ml) diluted in R10 medium were also added to test wells; 50 μl/well R10 medium and DMSO (Sigma) control were added to negative unstimulated wells and 50 μl/well Concanavalin A at 10□g/ml (Sigma) was added to positive control wells.

Plates were incubated at 37° C., 5% CO2 in a humidified incubator for 18-20 hrs. After extensive washing (1×PBS, 0.005% Tween) 50 ng/well of biotinylated anti-mouse IFN-□ antibody (U-Cytech) in PBS was applied. Plates were incubated for 2 hrs at RT, washed six times with washing buffer. Spots were developed by addition of development solution: 100 μl/well of streptavidin-alkaline phosphatase (PharMingen) and 1-Step NBT-BCIP (Pierce). Plates were incubated for 1 h at RT and washed again six times. Results are expressed as IFN-γ spot-forming units (SFU) per million of splenocytes counted using an ELISPOT counter (AELVIS). Background responses in media-only wells were almost always <20 spots and were subtracted from those measured in stimulated wells.

### RESULTS

### Production and Characterization of Anti-EphA2 and Anti ErbB2 IgG-OVA Antibodies

To generate anti-EphA2-OVA and anti-ErbB2-OVA fusion mAbs, we genetically fused DNA encoding the well-characterized H-2Kb-restricted CD8+ T-cell epitope from chicken ovalbumin OVA257-264 (SIINFEKL) or residues OVA253-267 (EQLESIINFEKLTEVV) to the CH3 C-terminal sequence of the anti-human EphA2 mAb 15 and the anti-ErbB2 Herceptin.

The resulting fusion proteins, nnAb15-OVAPep2 (SIINFEKL), nnAb15-OVAPep1 (EQLESIINFEKLTEVV) and Herceptin-OVAPep2 (SIINFEKL) were transiently expressed in HEK293-EBNA1 cell line. Wild type nnAb15 and Herceptin were also transiently expressed.

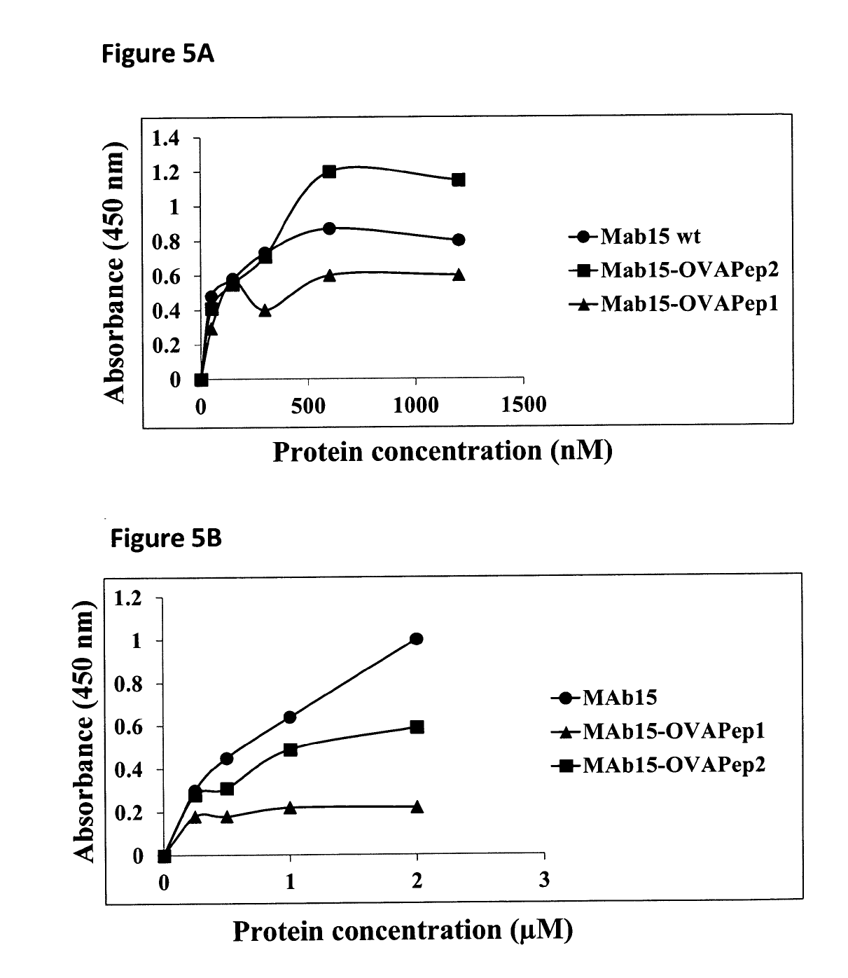

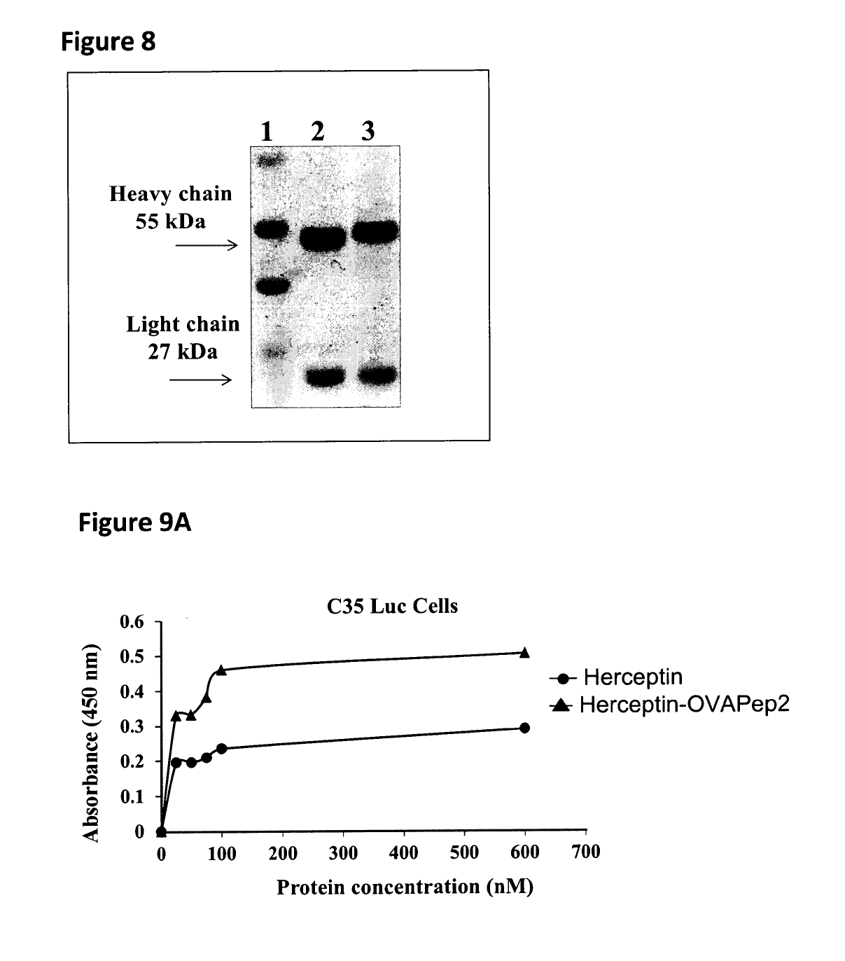

The encoded proteins, expressed as secretion products into the culture medium were harvested and purified by Protein A affinity chromatography. A one-step Protein A affinity chromatography yielded >99% pure proteins. Wild type and OVA-fusion antibodies showed comparable levels of expression. The visible proteins bands of approximately 55 and 27 kDa (FIGS. 3 and 8), represent the heavy and light chains of IgGs. As expected, Heavy chains of OVA fusion antibodies showed a slower migration with respect to the wild type Heavy chain.

Yields of the expressed mAbs were between 100 and 150 mg/L.

### Expression of the EphA2 Receptor on Cancer Cells

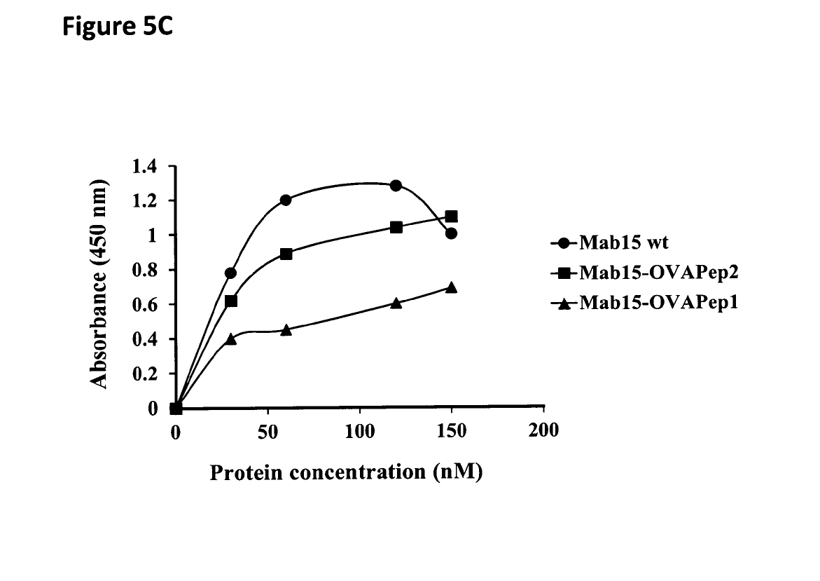

We evaluated the expression levels of EphA2 by western blot analysis in different tumor cells lines. We used 293/EphA2 as cell model of high expression levels. FIG. 4 shows expression levels of EphA2 in different cell lines. Human (HCT 116) and murine (TUBO, MC38 and 4T1) EphA2 positive cells expressed similar levels of EphA2 receptor.

Binding of Anti-EphA2 and Anti-ErbB2 mAbs to EphA2/ErbB2 Positive Cancer Cells Lines

Recombinant anti-EphA2 mAbs were tested for binding to the human EphA2 and for cross-reactivity with murine EphA2 displayed on cancer cell lines. Similarly, recombinant anti-human ErbB2 mAbs were tested for cross-reactivity with murine ErbB2 displayed on cancer cell lines.

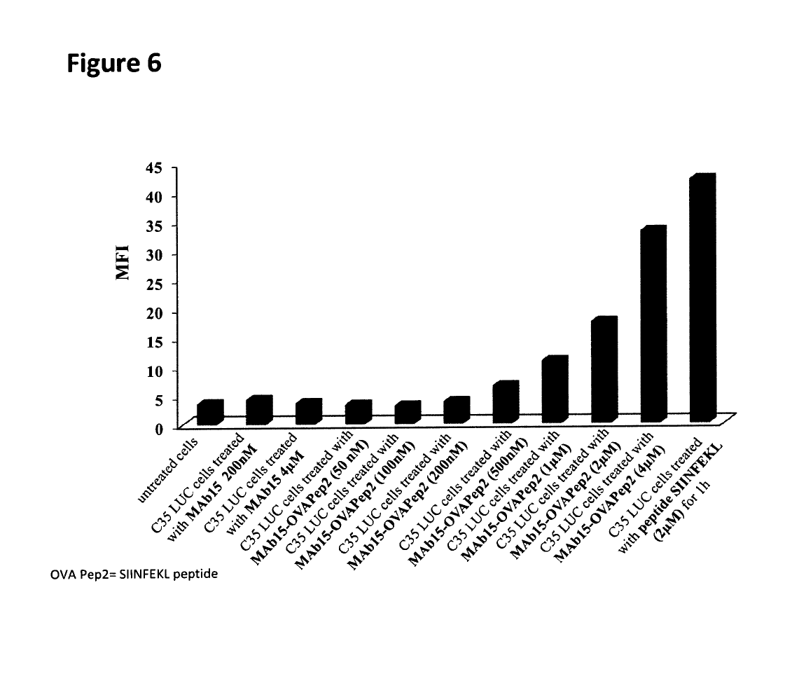

nnAb15-OVAPep2 and wt nnAb15 showed comparable binding to mouse and human EphA2-overexpressing cells lines (cell ELISA on murine C35luc and MC38 cell lines, and on human EBNA-293/EphA2). nnAb15-OVAPep1 showed a lower binding to EphA2-positive cells with respect to wild type nnAb15 (FIG. 5 A-C).

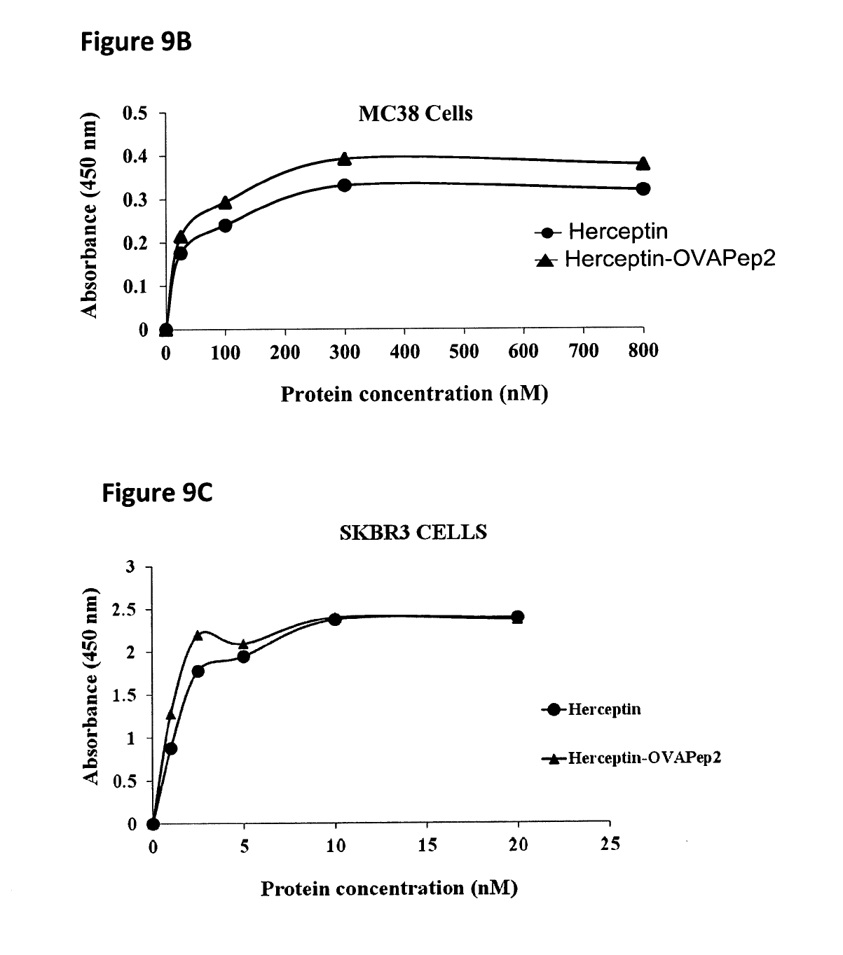

Herceptin-OVApep2 was found to retain a comparable affinity to that of the parental Herceptin for mouse and human ErbB2-overexpressing cells lines (cell ELISA on murine C35luc, MC38 and human SKBR3 cell lines) (see FIG. 9 A-C).

The binding of antibodies was detected with goat anti-human IgG1 peroxidase-conjugated antibody.

OVAPep2 is Presented on the Surface of the Tumor Cell in Complex with MHC-I

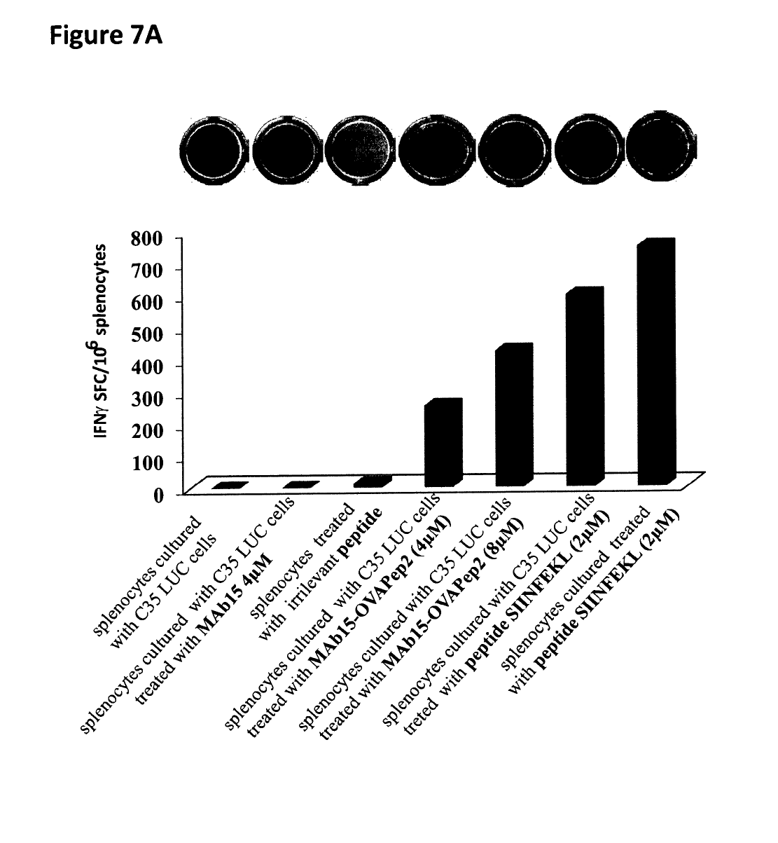

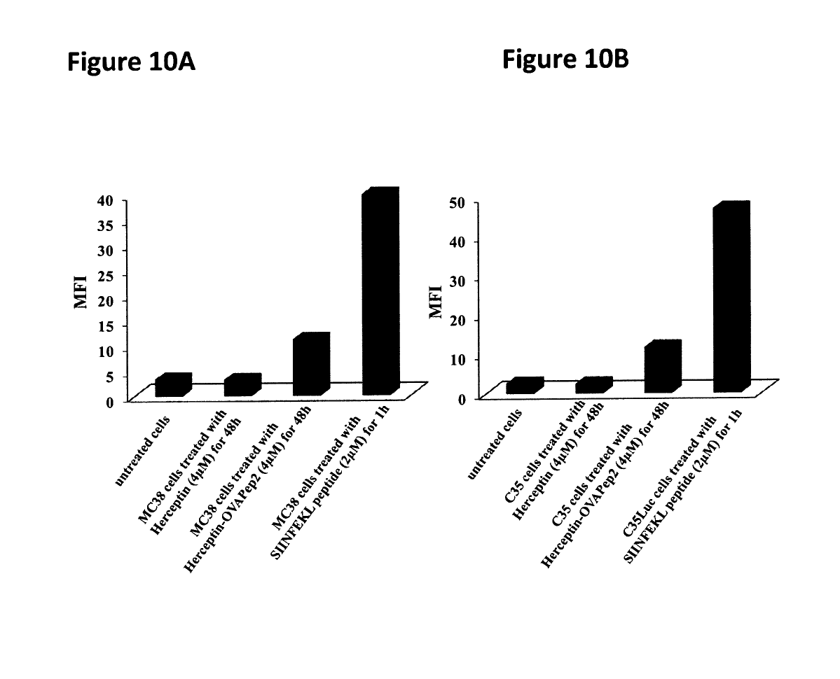

To verify whether EphA2- or ErbB2-expressing cells treated with anti-EphA2 or anti-ErbB2 fusion OVA mAb could present OVA257-264/Kb complexes on the cell surface, we incubated C35 luc and MC38 cells with increasing concentrations of antibody-OVA fusions. Cells were collected after 48 hrs and the surface OVA257-264/Kb complexes were detected by staining with specific mAb 25D1.16. As shown in FIGS. 6 and 10, incubation of cells with antibody-OVA fusions led to a dose dependent increase in mean fluorescence intensity (MFI) in the C35 Luc and MC38 cells.

These results indicate that the antibody-OVA fusions are efficiently internalized in target cells upon binding to their target molecules (EphA2 and ErbB2) and processed to release and present the OVA peptide (SIINFEKL) in complex with MHCI on the surface of the cancer cells.

OvaPep2 peptide/MHC-I complex on the surface of tumor cells is recognized by effector CD8 T cells induced by genetic vaccination

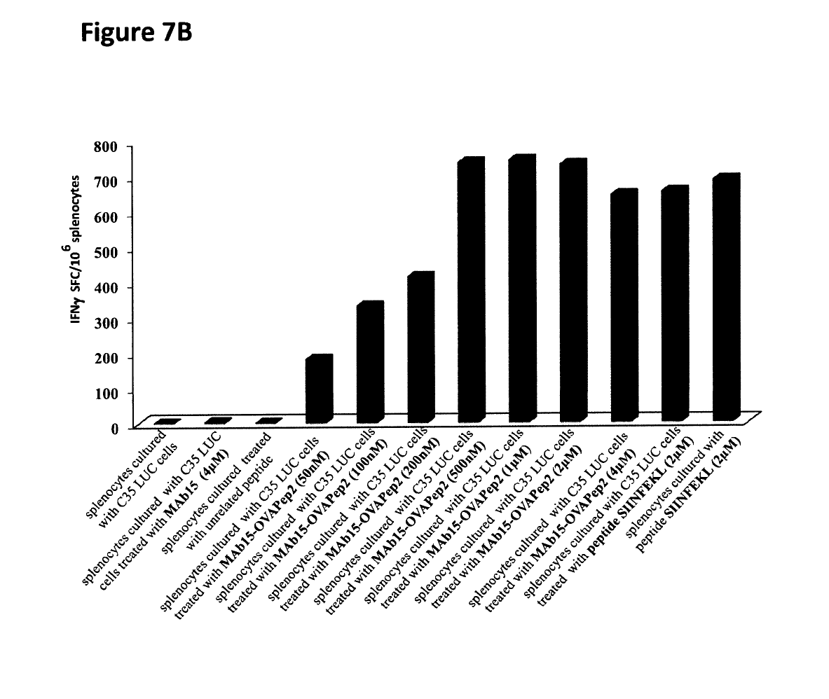

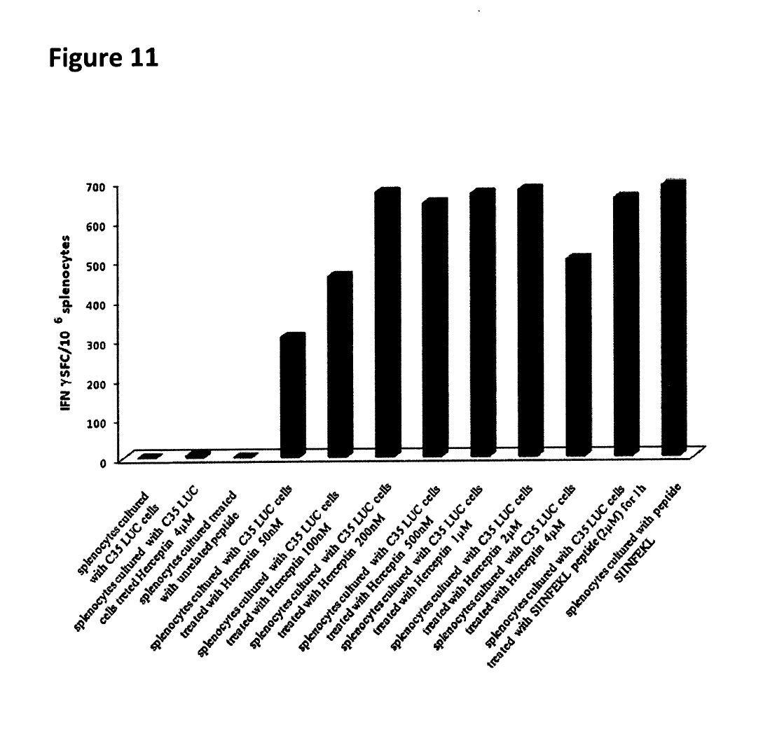

We determined whether binding of antibody-OVA fusions with consequent formation of OVA257 complexes on the surface of EphA2 and ErbB2 expressing tumor cells is recognized by OVA-specific CD8 T cells induced by genetic vaccination. To this end, we immunized mice with Ad5OVA TAG (encoding for the SIINFEKL peptide). Splenocytes from immunized mice (after 10 or 24 days from vaccination) were tested by IFN-γ ELISPOT upon incubation with C35 Luc cells treated with nnAb15-OVAPep2 or Herceptin-OVAPep2. Vaccine induced OVA-specific CD8 T cells specifically recognized and were activated by EphA2 or ErbB2 positive C35 Luc cells treated with nnAb15-OVAPep2 or Herceptin-OVAPep2 as shown by positive IFN-γ ELISPOT assay (FIGS. 7 and 11).

### CONCLUSION

In conclusion, in this study we have demonstrated that using a T-cell epitope fused to the CH3 C-terminal sequence of a mAb recognizing a tumor-specific cell surface receptor, the fusion antibody is internalized in target cells upon binding to the exposed tumor associated antigens and the T-cell epitope is efficiently presented in complex with the MHC-I on the tumor cells surface that becomes specific target of the cytolytic activity of vaccine induced CD8 T cells.

### Example 2

### Vector Mediated Presentation of OVA Epitope

We have shown that a TMEM according to this invention could be one where the targeting moiety is a viral vector (in this example we have used Adenovirus serotype 5-Ad5) and the Epitopic moiety is encoded in a nucleic acid (DNA) which is delivered into the target cell by the viral vector in a cell- and receptor-specific way.

Murine JAWSII cells (aplotype H-2Kb) were transfected with and expression plasmid encoding human coxsackie and adenovirus receptor (CAR). Plasmid DNA transfection was performed with Lipofectamine 2000 (Invitrogen) as described by the manufacturer. 48 hours after transfection, transfected and mock transfected cells were counted and 1-2×105 cells were seeded in 96 well plate (flat bottom plates; optimal if NUNC260860 plate). Cells were then infected with Ad5-OVA vector at an MOI of 200 (MOI calculated on ifu/ml) for 48 h.

After infection, cells were harvested, transferred into FACS tubes, washed with FACS buffer (PBS, containing 2 mM Hepes, BSA 2%) and centrifuged for 10 minutes at 1200 rpm.

Cells were incubated with 25 D1.16 antibody PE-conjugated, specific for the OVA257-264/Kb complex in the context of MHC class I.

After 1 h at RT and 1 hour at 37° C. with vigorous shaking, cells were washed twice with FACS buffer. Samples were resuspended in 200 μl of PBS and analyzed by flow cytometry using a Becton Dickinson calibur flow cytometer (BD Biosciences).

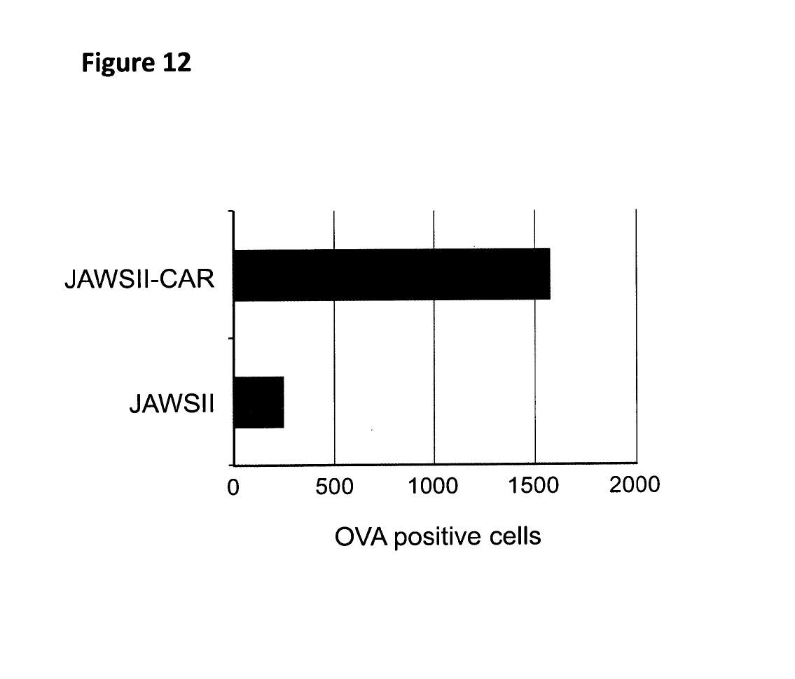

Results are shown in FIG. 12

The results indicated that Ad5-OVA infection of CAR expressing cells, but not car negative cells, led to elevated levels of OVA257-264 presentation.

1. A first composition comprising:
a ligand part being able to specifically bind to a target, wherein the target is on a diseased cell of an organism, and
an epitope part having a non-self epitope, or encoding a non-self epitope, wherein the non-self epitope is not naturally encoded by the organism, wherein
the target is internalized after forming a complex with the first composition and the non-self epitope is presented on the cell surface of said diseased cell after internalization.

2. The first composition according to claim 1 for use in the treatment of disease, wherein the first composition is used in conjunction with an immunogenic second composition comprising an epitope which is identical to the non-self epitope of the first composition, or comprising an epitope which elicits an immune response which is cross-reactive with the non-self epitope of the first composition.

3. (canceled)

4. (canceled)

5. A pharmaceutical composition comprising the first composition according to claim 1 and an immunogenic second composition comprising epitope which is identical to the non-self epitope of the first composition, or comprising epitope which elicits an immune response which is cross-reactive with the non-self epitope of the first composition.

6. A kit comprising:
a) a first composition according to claim 1; and
b) a immunogenic is second composition comprising an epitope which is identical to the non-self epitope of the first composition, or comprising an epitope which elicits an immune response which is cross-reactive with the non-self epitope of the first composition.

7. A composition according to claim 2 wherein the immunogenic second composition is delivered before the first composition or
after the first composition or
concomitantly, or substantially concomitantly with the immunogenic first composition.

8-14. (canceled)

15. A composition according to claim 1 wherein the target of the first composition is specific to a tumour cell, or has an expression pattern which is different from a non-diseased cell.

16-29. (canceled)

30. A composition according to claim 1, wherein said epitope part comprises one or more epitopes selected from the group consisting of Influenza virus epitopes, CMV epitopes, EBV epitopes, VZV epitopes, HCV epitopes, HBV epitopes, Measles virus epitopes, Rubella virus epitopes, rabies virus epitopes, yellow fever virus epitopes, epitopes from an antigen not associated with disease such as ovalbumin; and synthetic epitopes.

31. A composition according to claim 1, wherein said epitope part comprises an epitope against which for which a T cell immune response already exists in the organism.

32-41. (canceled)

42. A composition according to claim 2 wherein the immunogenic second composition additionally comprises an adjuvant.

43. A composition according to claim 42 wherein wherein the adjuvant is effective in promoting a cytotoxic T cell response.

44-45. (canceled)

46. The first composition of claim 1, wherein the ligand part is an antibody.

47. A method of treating cancer in a patient in need thereof comprising administering a first composition comprising a ligand part being able to specifically bind to a target, wherein the target is on a cancer cell in the patient; and
an epitope part having a non-self epitope, or encoding a non-self epitope, wherein the non-self epitope is not naturally encoded by the patient; wherein
the target is internalized after firming a complex with the first composition and the non-self epitope is present on the cell surface of said tumor cell after internalization.

48. The method of claim 47 wherein the ligand part is an antibody.

49. The method of claim 47 further comprising administering an in second composition comprising an epitope which is identical to or cross reactive with the non-self epitope of the first composition.

50. The method of claim 47 wherein the patient has previously generated a T cell response to said non-self epitope.

51. The method of claim 49 wherein the immunogenic second composition is administered before, at the same time as, or after the first composition.

52. The method of claim 49 wherein the immunogenic second composition additionally comprises an adjuvant.

53. The method of claim 52, wherein the adjuvant is effective in promoting a cytotoxic T cell response.

54. The method of claim 47 wherein the target of the first composition is specific to a tumor cell or has an expression pattern on a tumor cell that is different than on a non-tumor cell.

55. The method of claim 47 wherein said epitope part comprises one or more epitopes selected from the group consisting of Influenza virus epitopes, CMV epitopes, EBV epitopes, VZV epitopes, HCV epitopes HBV epitopes, Measles virus epitopes, Rubella virus epitopes, rabies virus epitopes, yellow fever virus epitopes, epitopes from an antigen not associated with disease such as ovalbumin; and synthetic epitopes.


SEQUENCE LISTING (7 sequences)

SEQ ID NO: 1 - protein, 132 aa, Influenza virus
NLNDTTYQRTRALVRTGMDPRMSSLMQGSTLPRRSGAAGAAVKGIGTMVMELIRMIKRGINDRNFWRGENGRKTRSAYERMANILKGKFQTAAQRAMMDQVRESRNPGNAEIEDLIFLARSALILRGSVAHK

SEQ ID NO: 2 - protein, 21 aa, Influenza virus
SNLNDTTYQRTRALVRTGMDP

SEQ ID NO: 3 - protein, 5 aa, Artificial Sequence
  Linker seqeunce
GGSGS

SEQ ID NO: 4 - protein, 15 aa, Gallus gallus
EQLESIINFEKLTEW

SEQ ID NO: 5 - protein, 8 aa, Gallus gallus
SIINFEKL

SEQ ID NO: 6 - protein, 16 aa, Artificial Sequence
  Secretory leader sequence
MGWSCIILFLVATATG

SEQ ID NO: 7 - protein, 9 aa, Artificial Sequence
  Control peptide. Sec is Selenocysteine
GAXQNEVTL